(19) United States
(12) Patent Application Publication
(10) Pub. No.: US 2006/0078540 A1
(43) Pub. Date: Apr. 13, 2006

(54) Dendritic cell nodes

(76) Inventors: William L Warren, Orlando, FL (US); Nir Hacohen, Cambridge, MA (US); Lan Bo Chen, Lexington, MA (US); Darell Irvine, Arlington, MA (US); Anatoly Kachurin, Orlando, FL (US); Russell G Higbee, Orlando, FL (US); Qian Huang, Arlington, MA (US)

Correspondence Address: MERCHANT & GOULD PC, P.O. BOX 2903, MINNEAPOLIS, MN, 55402-0903, US

(21) Appl. No.: 10/508,350

(22) PCT Filed: Mar. 18, 2003
(86) PCT No.: PCT/US03/08330

§ 371 Date: Sep. 26, 2005

(51) Int. Cl. C12N 5/08; A61K 48/00; A61K 35/14

(52) U.S. Cl. 424/93.1; 435/372; 424/422

(57) ABSTRACT

The present invention features dendritic cell nodes that can be used to vaccinate subjects against pathogens and to modulate a subject's immune system to treat or prevent various diseases and conditions.

### CROSS-REFERENCE TO RELATED APPLICATION

This application claims benefit of priority from Provisional Application Ser. No. 60/365,324, filed Mar. 18, 2002, which is herein incorporated by reference in its entirety.

### STATEMENT OF FEDERALLY SPONSORED RESEARCH

This invention was made with government support under Contract No. DAMD17-02-C-0130, awarded by the Defense Advanced Research Projects Agency (DARPA). The government may have certain rights in the invention.

### FIELD OF THE INVENTION

This invention relates generally to engineered dendritic cell nodes (DCN) that can be used to vaccinate subjects against pathogens and tumors and to modulate a subject's immune system to treat or prevent various diseases and conditions.

### BACKGROUND OF THE INVENTION

Dendritic cells (DCs) are involved in the initiation of both innate and adaptive immune responses. These “professional” antigen-presenting cells act cellular sentinels in every tissue of the human body, by detecting foreign antigens that serve as molecular signals of pathogen invasion.

During the adaptive immune response, an immature DC engulfs an antigen (e.g., an antigen from a pathogen, tumor, infected cell or other abnormal cell, or a self-antigen), after which the DC undergoes a maturation process and migrates to a lymph node. Over the course of this maturation process, the foreign antigen is cleaved into small peptides within the dendritic cell. These peptides are bound to major histocompatibility complex (MHC) class I and II molecules and presented on the surface of the mature dendritic cell. By presenting such processed peptides to T cells and B cells within the lymph node, mature dendritic cells directly and indirectly activate various subsets of these and other cells of the immune system, thereby guiding a series of immune responses that ultimately lead to elimination of pathogens.

Dendritic cells are not only critical for the induction of immune responses; they are also known to be important in the development of immune tolerance (e.g., to “self” antigens); when this process goes awry, autoimmune disease can result.

Infectious agents and tumor can evade endogenous dendritic cell surveillance through various mechanisms. To overcome these endogenous evasion mechanisms, therapies involving the injection of dendritic cells that have been stimulated with specific antigens ex vivo are being developed. For example, injections of antigen-stimulated dendritic cells have proven effective in animal models as both protective and therapeutic cancer vaccines. However, the first trials of dendritic cells therapy in humans have shown efficacy in only a small number of patients. In particular, it has been found that most of the injected dendritic cells die rapidly and fail to reach lymph nodes, and therefore, do not succeed in activating downstream T-cell and B-cells.

Accordingly, there is a need in the art for improved dendritic cell therapies.

### SUMMARY OF THE INVENTION

The present invention provides bioengineered dendritic cell nodes that can be used to modulate a subject's immune system. For example, the bioengineered dendritic cell nodes of the invention can be used to vaccinate a subject against one or more pathogens, to stimulate a subject's immune system against a tumor antigen for the treatment or prevention of cancer, or to tolerize a subject to an antigen (e.g., to treat or prevent allergies, asthma, autoimmune diseases, and rejection of transplanted cells, tissues, or organs).

In a first aspect, the invention features a dendritic cell node comprising a biocompatible scaffold material, a chemokine for attracting immature dendritic cells, a chosen antigen, and a maturation signal for dendritic cells.

In a second aspect, the invention features a dendritic cell node comprising a biocompatible scaffold material, a chemokine for attracting monocytes, a factor that induces differentiation of monocytes into immature dendritic cells, a chosen antigen, and a maturation signal for dendritic cells.

In a third aspect, the invention features a dendritic cell node comprising a first layer for attracting immature dendritic cells into the dendritic cell node, a second layer for presenting a chosen antigen to the immature dendritic cells, and a third layer for attracting dendritic cells and inducing maturation of dendritic cells.

In a fourth aspect, the invention features a dendritic cell node comprising a first layer for attracting immature dendritic cells into the dendritic cell node and for presenting a chosen antigen to the immature dendritic cells, and a second layer for attracting dendritic cells and inducing maturation of dendritic cells.

In a fifth aspect, the invention features a dendritic cell node comprising a first layer for attracting monocytes into the dendritic cell node, a second layer for inducing differentiation of the monocytes into immature dendritic cells, a third layer for presenting a chosen antigen to the immature dendritic cells, and a fourth layer for attracting dendritic cells and inducing maturation of dendritic cells.

In a sixth aspect, the invention features a dendritic cell node comprising a first layer for attracting monocytes into the dendritic cell node and for inducing differentiation of the monocytes into immature dendritic cells, a second layer for presenting a chosen antigen to the immature dendritic cells, and a third layer for attracting dendritic cells and inducing maturation of the dendritic cells.

The dendritic cell node of any of the above aspects of the invention can optionally comprise a symmetry layer. For example, the symmetry layer can be a second antigen presentation layer.

The dendritic cell node of any of the above aspects of the invention can optionally comprise a biocompatible encapsulating layer. For example, the encapsulating layer can be biodegradable, and can contain at least one bioactive substance to be released via diffusion from the encapsulating layer or via degradation of the encapsulating layer.

The antigen carried by the dendritic cell node of any of the above aspects of the invention can be a polypeptide, a peptide, a DNA molecule, or an RNA molecule.

In any of the above aspects of the invention, the dendritic cell node can optionally comprise cells. The cells can be autologous or non-autologous cells (e.g., but not limited to, monocytes or immature dendritic cells), which can be introduced ex vivo or in vivo. Immature dendritic cells can optionally be pulsed with antigen prior to being introduced into the dendritic cell node.

The dendritic cell node of any of the above aspects of the invention can be a folded construct, e.g., but not limited to, a four-quadrant folded construct. Alternatively, the dendritic cell node of any of the above aspects of the invention can be a rolled construct.

At least one layer of the dendritic cell node of any of the above aspects of the invention can comprise a polymer for sustained release of a factor embedded within the polymer. In one example, the factor can be within microspheres or nanoparticles, wherein the microspheres or nanoparticles are embedded within the polymer and undergo sustained release from the polymer.

The dendritic cell node of any of the above aspects of the invention can comprise at least one layer comprising bioconcrete, wherein the bioconcrete comprises a biodegradable mesh piercing a polymer gel.

In a seventh aspect, the invention features a method of constructing a dendritic cell node as described in any of the first six aspects of the invention. The method includes the steps of: a) depositing a first layer onto a substrate, and b) depositing each successive layer onto a proceeding layer, thereby constructing the dendritic cell node. Any of the dendritic cell nodes of the invention can be constructed in the sequential order of first layer to last layer, or in the reverse order, i.e., last layer to first layer.

For example, in an eighth aspect, the invention features a method of constructing a dendritic cell node. The method includes the steps of: a) depositing, onto a substrate, a layer for attracting immature dendritic cells into the dendritic cell node; b) depositing, onto layer (a), a layer for presenting a chosen antigen to the immature dendritic cells; and c) depositing, onto layer (b), a layer for attracting immature dendritic cells and inducing maturation of the immature dendritic cells. Alternatively, the method can include the steps of: d) depositing, onto a substrate, a layer for attracting immature dendritic cells and inducing maturation of the immature dendritic cells; e) depositing, onto layer (d), a layer for presenting a chosen antigen to the immature dendritic cells; and f) depositing, onto layer (e), a layer for attracting immature dendritic cells into the dendritic cell node, thereby constructing an dendritic cell node.

In a ninth aspect, the invention features a method of constructing a dendritic cell node including: a) depositing, onto a substrate, a layer for attracting monocytes into the dendritic cell node; b) depositing, onto layer (a), a layer for inducing differentiation of the monocytes into immature dendritic cells; c) depositing, onto layer (b), a layer for presenting a chosen antigen to immature dendritic cells; d) depositing, onto layer (c), a layer for attracting dendritic cells and inducing maturation of dendritic cells, thereby constructing a dendritic cell node.

The ninth aspect of the invention can further include the step of: e) depositing, onto layer (d), a layer for presenting a chosen antigen to immature dendritic cells, such that the dendritic cell node comprises two layers for presenting a chosen antigen to immature dendritic cells.

In a tenth aspect, the invention features a method of stimulating an immune response in a subject, comprising administering, to the subject, a dendritic cell node as described in any of the above aspects of the invention, wherein the dendritic cell node comprises an antigen and a dendritic cell maturation factor sufficient to stimulate an immune response against the antigen, thereby stimulating the immune response in the subject. The antigen can be e.g., from an infectious agent (e.g., a virus, a gram-negative bacterium, a gram-positive bacterium, a fungus, a protozoan, a rickettsium) or e.g., from a tumor cell.

In an eleventh aspect, the invention features a method of inhibiting an immune response in a subject, comprising administering, to the subject, a dendritic cell node as described in any of the above aspects of the invention, wherein the dendritic cell node comprises an antigen and a dendritic cell maturation factor sufficient to inhibit an immune response against the antigen, thereby inhibiting the immune response in the subject. For example, the antigen can be an allergen, a self-antigen (e.g., in autoimmune disease), or a non-self-antigen (e.g. on a non-autologous transplanted cell, tissue, or organ).

In a twelfth aspect, the invention features a method of attracting immature dendritic cells to a specific location within the body of a subject, comprising administering, to the subject, the dendritic cell node of the first, third, or fourth aspect of the invention.

In a thirteenth aspect, the invention features a method of attracting monocytes to a specific location within the body of a subject, comprising administering, to the subject, the dendritic cell node of the second, fifth, or sixth aspect of the invention.

In a fourteenth aspect, the invention features a method of slowing biodegradation of a polymer gel, comprising enclosing the polymer gel within a biodegradable mesh structure, thereby slowing biodegradation of the polymer gel. The polymer gel can contain a bioactive substance, in which case, the method slows release of the bioactive substance from the polymer gel. Moreover, the biodegradable mesh can optionally contain a bioactive substance to be released via diffusion from the biodegradable mesh or via degradation of the biodegradable mesh.

In a fifteenth aspect, the invention features bioconcrete, comprising a polymer gel carried within a biodegradable mesh. In one example, the bioconcrete can contain a bioactive substance within the polymer gel. In another example, the bioconcrete can contain a bioactive substance within the biodegradable mesh, wherein the bioactive substance is released via diffusion from the biodegradable mesh or via degradation of the biodegradable mesh.

In a sixteenth aspect, the invention features a method of preparing an antigen for uptake by a dendritic cell, comprising encapsulating the antigen within nanoparticles or microspheres, thereby preparing the antigen for uptake by a dendritic cell.

In a seventeenth aspect, the invention features a method of enhancing uptake of an antigen by a dendritic cell, comprising delivering the antigen packaged within nanoparticles or microspheres to the dendritic cell, thereby enhancing uptake of the antigen by the dendritic cell.

In any of the above aspects of the invention, the antigen can be a polypeptide, a peptide, a DNA molecule, or an RNA molecule. The antigen can also be a library of polypeptides, peptides, DNA molecules, or RNA molecules.

Additional advantages of the invention will be set forth in part in the description which follows, and those skilled in the art will recognize that other and further changes and modifications may be made thereto without departing from the spirit of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 is a diagram showing the architecture and various layers and components of an exemplary DCN.

FIG. 2(a)-(b) show, respectively, a photograph and a drawing of the biological architecture tool (BAT).

FIG. 3 is a diagram showing the chemical composition of hyaluronic acid.

FIG. 4 is a depiction of two photographs showing a pyramid-shaped, collagen/gelatin engineered tissue construct (ETC) containing eight layers.

FIG. 5 is a depiction of two photographs displaying a vehicle (left panel) and capsule (right panel) built with PF-127/PPF-PEG mix.

FIG. 6(a)-(g) is a depiction of a series of photographs showing: (a) layer-by-layer construction of a capsule; (b) filling the capsule with various layers of the DCN; (c) a filled capsule; (d) rinsing the filled capsule in saline and cutting it off the slide; (e) fitting the filled capsule into an injection needle; (f) close view of capsule in needle; (g) subcutaneous injection of capsule into a chicken.

FIG. 7(a)-(b) is a depiction of two photographs showing mesh forms fabricated by the BAT; (a) shows a two-layer PPF “log cabin”; (b) shows a four-layer PCL mesh.

FIG. 8(a)-(c) is a depiction of three photographs showing a viability test in a test-well constructed using the BAT and the compositions and methods of the invention. (a) shows a PF-127/PPF-PEG test-well filled with fibrin glue; (b) shows fibroblasts deposited together with thrombin into the test-well; (c) shows the fibroblasts after a 48-hour incubation at 37° C.

FIG. 9 is a diagram showing three strategies for controlled release from the DCN: (1) cross-linked networks; (2) controlled release microspheres; and (3) controlled release nanoparticles.

FIG. 10(a)-(b) is a pair of graphs showing controlled release of proteins from: (a) triblock hydrogels encapsulating bovine serum albumin; and (b) PLGA/PEG microspheres encapsulating ovalbumin.

FIG. 11(a)-(b) respectively show: (a) an NMR spectrum showing the structure of a PGLA-PEG-PLGA triblock copolymer (arrows and shading indicate the corresponding resonances from the schematic structure); and (b) a graph showing the results of a triblock hydrogel toxicity assay (100 mg of PGLA-PEG-PLGA was photo-polymerized in one culture well; on Day 7, bone marrow-derived dendritic cells were added to the well with the gel (solid bars) or to the controls (open bars) and were cultured for 24 hours). FIG. 12 is a series of panels relating to drug delivery components: (a) is a depiction of an optical micrograph (OM) showing protein-loaded PLGA microspheres; (b) is a schematic of PLGA-PEG-PLGA-based hydrogel nanoparticles; (c) is a depiction of a scanning electron micrograph (SEM) showing nanoparticles; (d) is a depiction of an ethidium bromide-stained gel showing DNA recovered from biodegradable nanoparticles lysed with 0.1 M NaOH; (e) is a depiction of a pair of photomicrographs (left=brightfield, right=fluorescence) of dendritic cells containing phagocytosed nanoparticles.

FIG. 13 is a chart showing various factors to consider when choosing biomaterials for the dendritic cell node.

FIG. 14 is a representation of a photomicrograph showing fMLP droplets close-up on a scaffold patch.

FIG. 15 is a representation of a photomicrograph of fMLP droplets deposited on a scaffold patch, which shows that the scaffold margins are free of droplets.

FIG. 16 is a depiction of a pair of photomicrographs showing triblock gel particle uptake by dendritic cells after two hours in culture (left=bright field; right=fluorescence).

FIG. 17 is a depiction of an ethidium bromide-stained gel showing DNA encapsulation in degradable nanogel particles.

FIG. 18 is a graph showing attraction of immature dendritic cells to fMLP peptide.

FIG. 19 is a depiction of the results of a microarray analysis showing gene expression in human monocyte-derived dendritic cells.

FIG. 20 is a graph showing a strategy for producing a dendritic cell node with a folded quadrant structure.

FIG. 21 is a diagram showing a strategy for producing a dendritic cell node with rolled layers.

### DETAILED DESCRIPTION OF THE INVENTION

The present invention provides dendritic cell nodes (DCN) and methods for making and using the same. The DCN, as described herein, is an implantable, three-dimensional (3D), tissue-engineered (TE) scaffold that can be used to modulate (increase or decrease) the immune responses of a subject. Accordingly, the DCN can be used to stimulate the immune system, e.g., to vaccinate against infectious agents or to treat or prevent cancer. The DCN can also be used to tolerize against antigens, e.g., to treat or prevent allergies, asthma, autoimmune disease, or rejection of transplanted organs, tissues, or cells.

The DCN is an engineered tissue construct (ETC) that contains base scaffold materials and biomolecules. The term “base scaffold materials” refers to the biomaterials used to construct the ETC, such as (but not limited to) collagen, fibrin glue, hyaluronic acid (HA), triblock copolymers, poly(lactide-co-glycolide) (PLGA). Biomolecules include, e.g., chemicals, vitamins, hormones, molecules, proteins, nucleic acid molecules (e.g., plasmid or viral vectors), antigens, chemokines, and cytokines, that are located within the base scaffold material to induce a specific response and/or functionality. In addition, the DCN can optionally be populated with cells during its fabrication.

Abbreviations and symbols used throughout this specification are set forth in Table 1.

Dendritic Cells

The human body's immune system is a complex and potent network, the adaptability of which is mediated by several key cell types, the most important of which are dendritic, T, and B cells. Toll-like receptors (Tlr) are believed to be the first line of recognition at the time of pathogen encounter (Takeda K, Kaisho T, Akira S. Toll-like receptors, Annu Rev Immunol. 2003; 21:335-76). DC's, which are the most potent antigen-presenting cells (APC's) known, express a large number of the ten known Tlr genes and can be used to develop novel TE vaccines.

DC's serve as cellular sentinels, standing guard in every tissue of the human body, ready to detect the antigens that are the molecular signs of pathogen invasion. DC's initiate both adaptive and innate immune responses (Ref. 1). They are the most powerful APC type; they ingest antigens at infection sites and present them in lymphoid organs to T cells as peptides bound to both Major Histocompatibility Complex (MHC) class I and II products. DC's initiate and control the quality of the T-cell response, driving the transformation of naïve lymphocytes into distinct classes of antigen-specific effector cells. In addition, DCs directly stimulate the adaptive B cell responses (Litinskiy M B, Nardelli B, Hilbert D M, He B, Schaffer A, Casali P, Cerutti A. DCs induce CD40-independent immunoglobulin class switching through BLyS and APRIL. Nat Immunol. 2002 September; 3(9):822-9; Craxton A, Magaletti D, Ryan E J, Clark E A. Macrophage- and dendritic cell-dependent regulation of human B-cell proliferation requires the TNF family ligand BAFF. Blood. 2003 Jan. 16 12531790; MacLennan I, Vinuesa C. Dendritic cells, BAFF, and APRIL: innate players in adaptive antibody responses. Immunity. 2002 September; 17(3):235-8; Schneider P, MacKay F, Steiner V, Hofmann K, Bodmer J L, Holler N, Ambrose C, Lawton P, Bixler S, Acha-Orbea H, Valmori D, Romero P, Werner-Favre C, Zubler R H, Browning J L, Tschopp J. BAFF, a novel ligand of the tumor necrosis factor family, stimulates B cell growth. J Exp Med. 1999 Jun. 7; 189(11):1747-56.) DC's are also critical players in innate immunity. They produce cytokines important to host defense and to activation of natural killer cells (NKC's) that kill target cells and produce important cytokines (Ref. 2).

Before leaving the lymph node, T cells also activate B cells (in synergy with the indirect and direct effects of dendritic cells on B cells), which then produce antibodies that bind to pathogens or to their toxic products and prevent their harmful effects. Dendritic, T, and B cells also recruit other classes of immune cells to participate in thwarting an invading pathogen. Effectively, DC's trigger and guide a chain reaction of immune responses that leads to elimination of a pathogen.

Described herein are bioengineered, DC-activating ETC's, containing DC's or not, that transmit molecular signals to activate the body's DC's, which can be released and then typically either migrate to the natural host lymph nodes; or mature and entice T cells to enter and trigger further immune responses at the site of vaccination. The two general approaches to DCN construction are as described in Table II.

In a first example, TE scaffolds are not populated with DC's during fabrication, but are endowed with (a) chemokines that attract immature DC's (iDC's) or monocytes; (b) the pathogenic antigen(s); (c) various DC modulators, as will be discussed later for immunity; and/or (d); suppressors for immune tolerance to induce mature DC's to migrate from the DCN to “natural/host” draining lymph nodes after programming and antigen-loading has occurred.

This DCN embodiment is an implantable DC docking vaccine; this type of DCN includes the ability to concentrate a large number of DC's in a small area subcutaneously. These DCNs can include appropriate antigens for the pathogen, for example, using recombinant proteins or peptides (or libraries thereof), DNA molecules (e.g., plasmids, viral vectors, etc.) or RNA molecules that encode the desired antigen (or libraries thereof), and appropriate state inducers to program the optimal response for a pathogen and to induce DC's to migrate from the DCN to “natural/host” draining lymph nodes after antigen loading and programming has occurred.

A porous ETC is created that can release factors with fine control—concentration and start/end times using biodegradable microspheres or by appropriately embedding the biomolecular factors in the scaffold host material—in the same way that the body does during a response.

In a second example, ETC's can be populated with DC's during fabrication. Controlled exposure to signaling molecules (e.g., cytokines and chemokines) together with engineered antigens (based on pathogens' molecular components) in an ETC allow optimal activation of DC's so that a powerful immune response is initiated. For either type of DCN (fabricated with or without DC's), afterwards, these constructs are subcutaneously injected into the patient prior to tumor and/or pathogen challenge. The best scaffold, microenvironment, gradients, and concentrations are optimized, all of which are provided by the tools and methods disclosed herein. Table III provides examples of ligands for use in modulation of DC's on the scaffold.

In vivo attraction and repulsion of DC's has been shown by the successful attraction of iDC's to subcutaneously implanted polymer rods (Ref. 3). These DC's were loaded with a tumor-associated antigen and naturally emigrated, repelled from the rods and were found to home to lymph nodes (Ref. 4). The 3D scaffolds described herein not only allow the attraction and repulsion of DC's, but also the selection for optimal DC subtypes and the modulation of their maturation state to maximize the efficiency of antigen presentation to the immune system.

Effective DC-based immunotherapies are developed through the rational manipulation of DC's with scaffolds and deposition, and, various modulators to maintain their proper activation and maturation states, enhance their viability, and facilitate their migration to lymph nodes. Disclosed are artificial TE dendritic cell nodes that can be repackaged for cures for diabetes, arthritis, lupus, cancer, infectious disease, autoimmune diseases (such as Type I Diabetes, Lupus, rheumatoid arthritis, multiple sclerosis and others). The DCN can be redesigned to target one disease at a time by controlling the maturation states of the DC's and/or loading them with the proper antigen(s) associated with the target antigen of interest. Furthermore, the DCN can also develop a TE scaffold for inducing tolerance, because the DC is involved in tolerance. It is then possible to address a vast number of inflammatory diseases, including autoimmunity, allergy, and asthma.

Dendritic Cell Properties

As mentioned above, DC's protect human tissues by detecting the antigens that are the molecular signs of pathogen invasion. DC's are APC's with a unique ability to induce primary immune responses. DC's capture and transfer information from the outside world to the cells of the adaptive immune system. DC's can initiate both adaptive and innate immune responses (Ref. 5). DC's are not only critical for the induction of primary immune responses, but may also be important for the induction of immunological tolerance, as well as for the regulation of the type of T-cell-mediated immune response.

DC's initiate an immune response in various ways. Immature DC's can directly interact with pathogens that induce the secretion of cytokines. e.g., interferons (IFN's), which in turn can activate the immune system. After capturing antigens, iDC's migrate to lymphoid organs (e.g., lymph nodes) where they mature. After maturation, they display peptide MHC's, thereby enabling the selection of rare circulating antigen-specific lymphocytes. Thus, DC's initiate and control the quality of the T-cell response, driving the transformation of naïve lymphocytes into distinct classes of antigen-specific effector cells. Activated T cells are able to migrate and reach the diseased tissue. Helper T cells (CD4⁺ T cells, Type I; symbol T_{H}1) secrete cytokines, which permit activation of macrophages, NKC's, and cytotoxic CD8⁺ T cells. Cytotoxic T cells eventually lyse (kill) the diseased or infected cells. Specifically, CD8⁺ T cells directly kill the tumor or pathogen. Other T-helpers (of Type II; symbol T_{H}2) activate B cells, which produce antibodies that bind to pathogens or to their toxic products, thereby preventing their access to cells. Using the cytokine network, dendritic, T, and B cells also recruit other classes of immune cells to participate in thwarting an invading pathogen. Effectively, DC's trigger and guide a chain reaction of immune responses that leads to elimination of a pathogen.

From the aforementioned chain of events, it has been hypothesized that DC's are a link between innate immunity and adaptive immunity in antitumor immune responses (Ref. 6-7).

Immune Response Evasion Mechanisms

Even though DC's are a key component of immunological strategies, infectious agents and tumors can evade DC surveillance through several mechanisms. Certain agents may not produce inflammation, which normally facilitates antigen uptake by DC's. Some microorganisms might restrain DC's by producing inhibitory molecules (Ref. 8). To address these evasive mechanisms, therapies based on the injection of DC's, charged with antigens ex vivo, are being actively developed.

Dendritic Cell Therapy

In the field of cancer treatment, DC-based treatments have demonstrated regression of tumors. Tumor-specific antigens are presented to DC's in controlled conditions outside the body; these antigen-loaded DC's are then injected to initiate an immune response. In animal models, DC therapy has proven effective both as cancer vaccines and immunotherapy. Injection of bone-marrow-derived DC's pre-pulsed with tumor-associated peptides has been shown to protect mice against subsequent lethal tumor challenge (Ref. 9). Moreover, in mice bearing established macroscopic tumors, treatment with tumor-peptide-pulsed DC's resulted in sustained tumor regression and tumor-free status in 80-100% of cases (Ref. 9-10). Similar results have been observed with the injection of tumor lysate-pulsed DC's in mice (Ref. 7). The injection of DC's charged with tumor-associated antigens (Ref. 9-11) has proven effective in animal models both as protective cancer vaccines and as therapies to eliminate preexisting tumors.

Dendritic Cell Vaccination Results in Humans

Injections of DC's charged with antigens (Ref. 9-11) have proven very effective in animal models as both protective and therapeutic vaccines as discussed above. However, the first trials of DC therapy in humans have only shown efficacy in a small number of patients (Ref. 12-13). Whereas numerous factors might be involved in the treatment's low efficacy, a consistent finding has been that most of the DC's died upon injection. Because of improper maturation, very few (0.1%) DC's reached the natural lymph nodes. Improvement of this therapy has recently been demonstrated in animal studies when DC viability, activity, and state are enhanced by turning on certain genes in DC's by modulators (Ref. 14-15). In addition, recent human trials with DC vaccination for influenza have clearly demonstrated the importance of the DC activation and maturation states in eliciting potent responses (Ref 3, 16).

Why Use Engineered Constructs?

The present invention provides TE scaffolds as a means to overcome specifically the aforementioned obstacles in DC-based vaccines. TE scaffolds provide the following attributes as they pertain towards the DCN for vaccine discovery:

Scaffolds endowed with appropriate biomolecules (cytokines) will help to extend the life of the DC's and to activate and mature them appropriately, thus enabling a more potent effect with fewer injections.

Targeted antigens for presentation by DC's are controlled by TE scaffolds.

State modulators of the DC's are controlled by incorporation of these ligands in the TE scaffold.

Dendritic Cell Node Overview

The DCN is an ETC that can be introduced (e.g., subcutaneously) into a human or other animal. The DCN contains various chemoattractant layers that, variously: (1) attract endogenous monocytes (or other DC precursors) from the host animal in which the DCN is implanted, (2) induce differentiation of the host monocytes into immature DC's, (3) load the immature DC's with specific antigens, and (4) induce maturation of DC's, which then migrate to a draining host lymph node. At the endogenous host lymph node, the mature DC's activate endogenous pre-programmed naive T and B cells (the ones matched for the antigen from the large repertoire of T and B cells). The natural host lymph node is the location where of T and B cells reside and find their matched antigen.

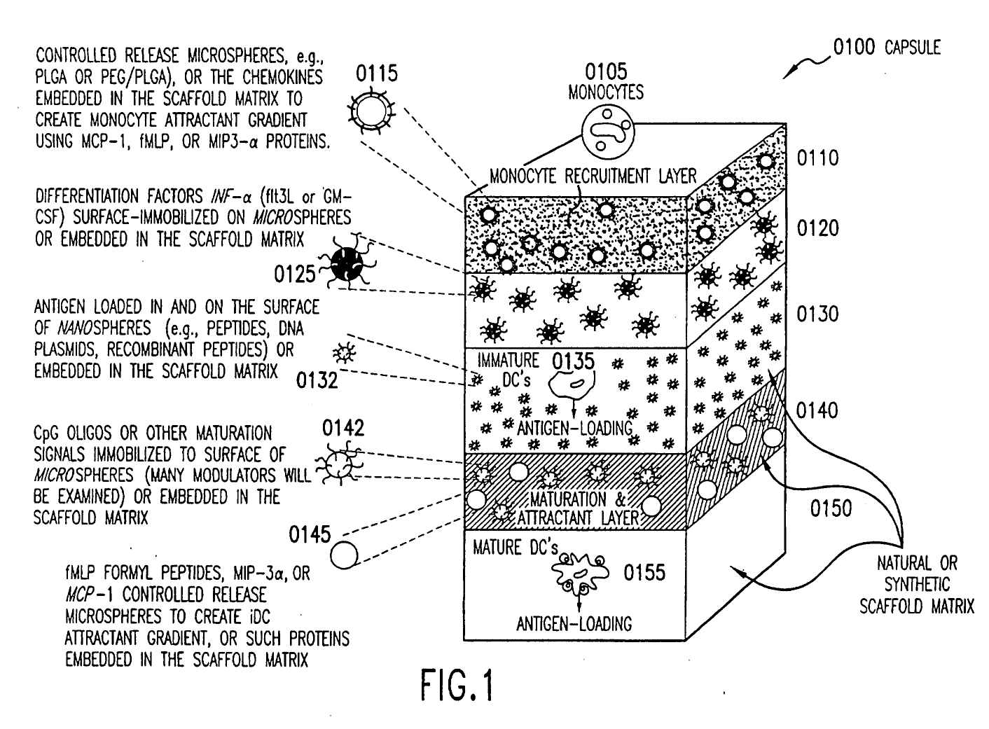

The DCN, as shown in FIG. 1, has the abilities to: (1) differentiate monocytes 0105 to iDC's 0135; (2) attract both monocytes 0105 and DC's 0135 and 0155 alike via chemotactic layers; (3) load antigens 0132 onto the iDC's 0135; and (4) differentiate these iDC's 0135 into mature DC's 0155 both in vitro and in vivo. Various different antigens 0132 associated with a number of diseases, e.g., (but not limited to) cancer, diabetes, human immunodeficiency virus (HIV), malaria, can be used. Other permutations to achieve the DCN functionality are also possible. For example, the DCN can be constructed in such a way that functions of several of the layers are combined; only three layers are necessary, with the three layers being an antigen-presenting layer, a maturation signal layer with appropriate ligands, and an antigen-presenting layer with a DC chemokine in all three layers. In this case, the monocyte recruitment layer 0110 and/or the differentiation layer 0120 is not included, as the DCN simply attracts DC's already in the body.

To build biocompatible structures that replicate or enhance the natural living system (microenvironment, 3D structure, chemotactic gradients, etc.) to support cell development, the disciplines of digital manufacturing, tissue engineering, and immunology are incorporated to create the DCN. The digital printing computer-aided-design/computer-aided-manufacturing (CAD/CAM) techniques of the Biological Architectural Tool (BAT) are used to build designer 3D heterogeneous ETC's; however, in principle, other digital printing tools may also be used.

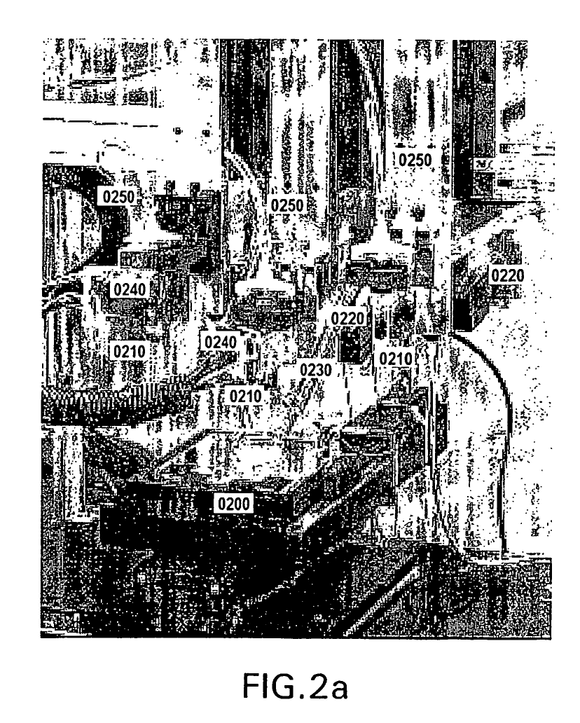

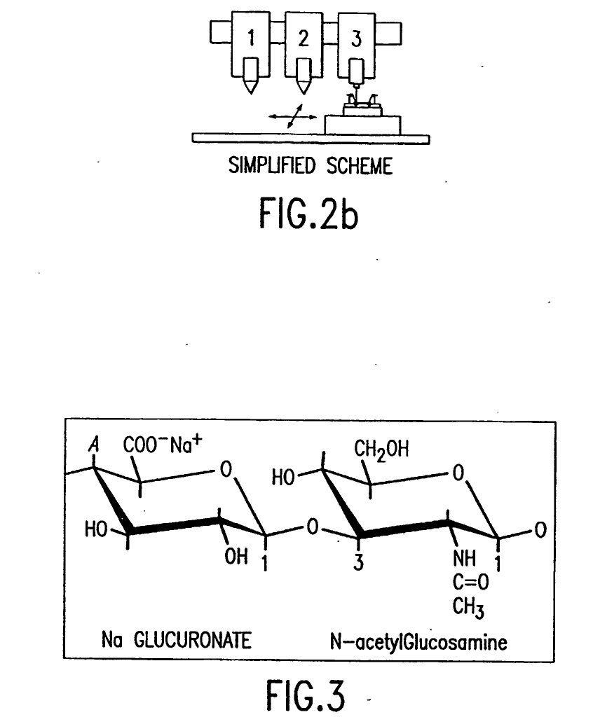

The BAT is a 3D, multiple-head, through-nozzle printing machine, shown in FIG. 2, which can be used to directly deposit the components of the DCN, such as biomaterials, cells, and molecular cofactors (the BAT is described in detail in PCT/US02/26866, herein incorporated by reference in its entirety for its teachings regarding how to make and use the BAT). Examples of such biomaterials, cells, and molecular cofactors include, but are not limited to:

Biomaterials: collagen, ECM materials, fibrinogen, thrombin, fibrin glue, HA, PLGA, PPF-PEG, PCL, gelatins (including photocurable gelatins), Pluronic F-127, triblock A-B-A (e.g., PLGA-PEG-PLGA dimethacrylate) copolymers.

Cells: endothelial, epithelial, dendritic, T, and B cells; monocytes, macrophages, neurons, fibroblasts, stem cells.

Molecular Cofactors: cytokines, chemokines, DNA plasmids, libraries of expressed antigens, proteins, glycoproteins, peptides, vitamins.

These materials are deposited onto various supporting substrates and surfaces to create surrogate tissues and experimental platforms for experiments in cell biology and tissue engineering. The BAT deposits the DCN and other ETC's in a layer-by-layer (LBL) mode. The device (FIG. 2(a)) consists of an xyz coordinate stage 0200; a number of microdispensing deposition heads or pens 0210, each of which has an individual observation and tuning video camera 0220; a light source to cure photopolymers in-line 0230; a system of individual temperature control for the pens and the stage 0240; compressed air to pressurize pens 0250; a humidifier preventing dehydration of living samples; and a computer controlling the whole deposition process (the latter two not shown). The BAT has been designed as an upgradeable system, allowing more units and functions to face upcoming tasks to be built therein.

Base Scaffold Materials Used to Fabricate the DCN

Next are discussed candidate base scaffold materials for constructing the DCN in LBL mode using a digital printing apparatus, i.e., the biomaterials. Later, the role and ingredients (biomolecules) of each layer in the entire construct is presented in detail; i.e., the various molecular factors that are added to each layer in the multilayer DCN. The synthetic and natural polymers (biomaterials) shown in Table IV are only representative. Other biomaterials and configurations can also be used. Below are presented several specifics regarding a few of the candidate scaffold materials.

Base Scaffold Biomaterials

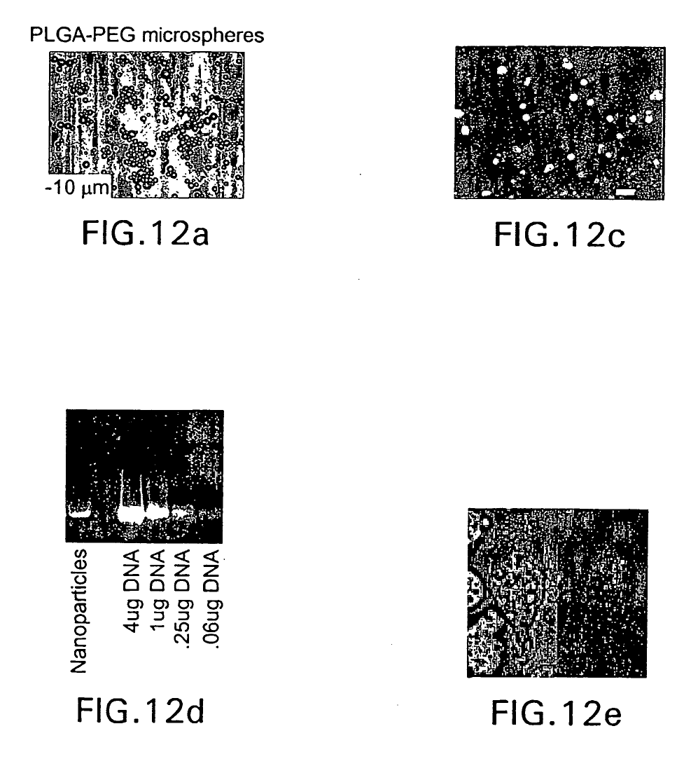

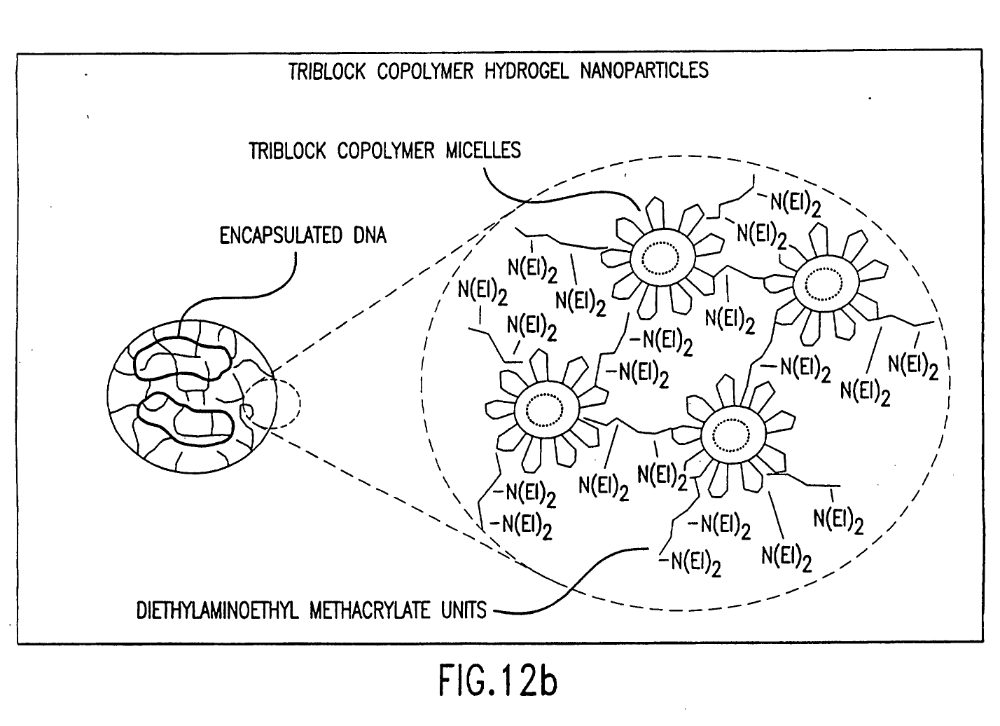

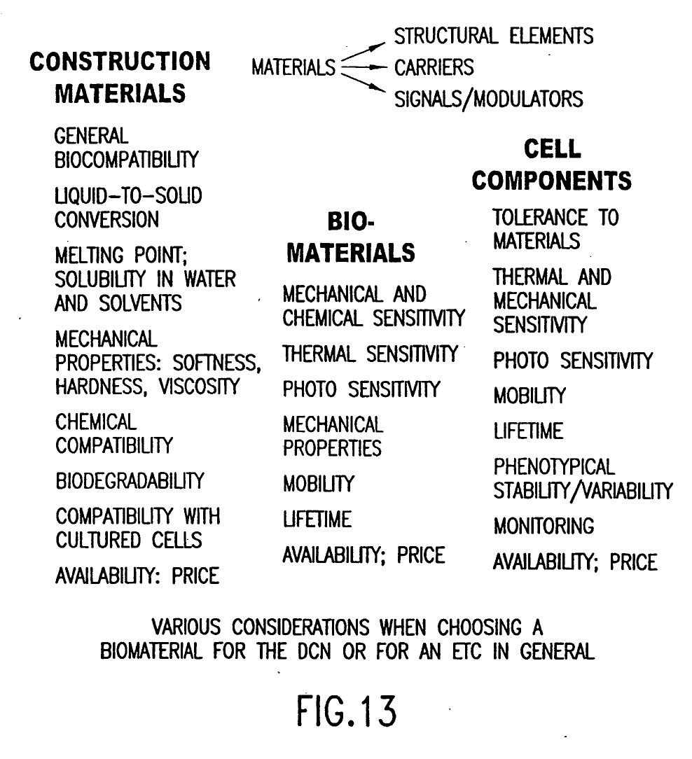

Biomaterials as set forth in Table IV can be used to construct the base scaffolds and associated capsules of the DCN. The base scaffold biomaterials simply need to be of good construction properties (retain their shapes), and be biocompatible and biodegradable, etc., as shown in FIG. 13.

Fibrin Glue

This fibrinogen-thrombin-calcium(II) system produces stable clots firmly attached to various surfaces. This system can be combined with natural components like HA and collagen, thus providing the necessary stickiness and stability of gel layers in aqueous solutions. Several fibrin glue patches containing laminin have been fabricated for cell viability and have shown promising results. One particular fibrin biomaterial configuration is detailed below. The following description is exemplary only, as other combinations can be used without departing from the spirit and scope of the invention.

These fibrin glue patches were 5×5 mm squares deposited in 30-mm plastic Petri dishes, one patch per dish. The patches were deposited in LBL mode using two different solutions: (1) Solution “Fibro” contained 80 mg/mL fibrinogen and 0.1 mg/mL laminin in distilled water; (2) Solution “Thrombo” contained 22 mg/mL thrombin in a solution containing 20 mM CaCl₂ and 1% w/w HA.

Samples:

A1-A6: “Fibro” deposited first, “Thrombo” second.
B1-B2: Same as A series, except “Fibro” reduced about 30%.
C1-C3: “Thrombo” deposited first, “Fibro” second.
D1-D4: Same as C series; the deposition rate for “Fibro” was reduced 3× while total quantities were kept the same.
   Estimated Loading of Components in the Patches:
Laminin: 6±2 μg/cm²
Fibrin clot: 3±1 mg/cm²
HA: 0.40±0.15 mg/cm²

The foregoing description of fibrin glue patches is an example only and does not limit the concentrations of ingredients used in such patches. For example, the fibrinogen concentration can be from about 0.1 mg/ml to about 100 mg/ml, e.g., about 0.1 to about 1 mg/ml, about 1.0 to about 10 mg/ml, or about 10 to about: 20, 30, 40, 50, 60, 70, 80, or 90 mg/ml. The thrombin concentration can be about 0.1 mg/ml to about 30 mg/ml, e.g., about 0.1 to about 1 mg/ml, about 1.0 to about 10 mg/ml, or about 10 to about 20 or about 30 mg/ml.

Hyaluronic Acid: A Universal Thickening Additive

Hyaluronic acid (HA) is a universal component of the extracellular spaces of body tissues. This mucopolysaccharide has an identical chemical structure whether it is found in bacteria or human beings. It is composed of repeating disaccharide units of N-acetylglucosamine and D-glucuronic acid as shown in FIG. 3.

HA retains significant amounts of water to form a liquid gel. HA increases the viscosity of fluids, thus facilitating control and improving quality of deposition for cellular suspensions as one example.

HA is miscible with any synthetic or natural material listed in Table V without side effects. Being a natural component of the ECM material, it is harmless to cells. Preliminary results indicate that a 1% solution of HA supports the suspension of cells for days, preventing early agglomeration. Thus, this should be an ideal biomaterial component for such ETC's as the DCN.

Generally, the fibrin glue and HA additives to such natural polymers as collagen and ECM show significantly improved construction/building properties, allowing the ETC to be built in LBL mode.

Collagen and Gelatin Layers

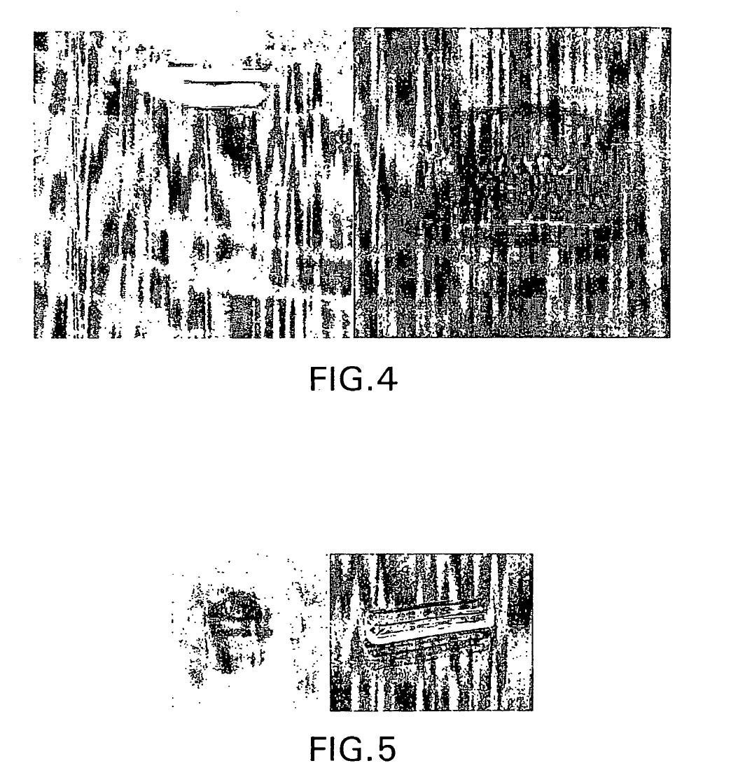

Collagen and gelatin layers also make promising scaffold materials. FIG. 4 shows photographs of an alternating collagen/gelatin eight-layer pyramid construct. The gelatin has greater construction properties; however, the collagen shows improved construction upon adding fibrin glue and HA to the scaffold matrix. Both the collagen, gelatin, HA, and ECM natural polymers are soluble in bodily fluids and can degrade quickly. Methods are disclosed below on how to decrease the degradation rate of these natural polymers using bioconcrete.

PF-127

The use of PF-127 in combination with PPF-PEG (22%-25% and 12%-10% solutions in phosphate-buffered saline (PBS), respectively) allow the building of sophisticated 3D constructs, including closed boxes and capsules stabilized by photo-crosslinking of PPF-PEG as shown in the next section. In general, PF-127 mixed with other viscous components retains its remarkable shape-forming capacity, but only to a limit. When the share of the other component exceeds a certain level, the solution will likely lose the feature of reverse-temperature gelation intrinsic to PF-127 and turn into a primitive, viscous syrup.

Injectable Capsule Made of PF-127/PPF-PEG Combination

DCN constructs comprising a number of layers of combined natural and synthetic materials can be encapsulated in a miniature vehicle, the material of which can act like an antigen or cytokine depot carrier as well. Hard gelatin, e.g., can be used for this task. The injectable capsule can serve as a temporary “housing” for the proper DCN ETC. The capsule in this case is used to withstand the shear forces upon injecting the DCN ETC in the patient via subcutaneous injection.

As one example, a combination of PF-127 with PPF-PEG provides excellent 3D printing and stability in aqueous environments due to photo-crosslinking of the PPF-PEG component. FIG. 5 shows a vehicle and a capsule built with the PF-127/PPF-PEG mixture. The box measures 5×5×2 mm; the capsule is 7×1.4×0.8 mm. PF-127 has been successfully used for controlled subcutaneous delivery of drugs, including insulin. It could probably alleviate any possible negative effects of PPF-PEG on cells.

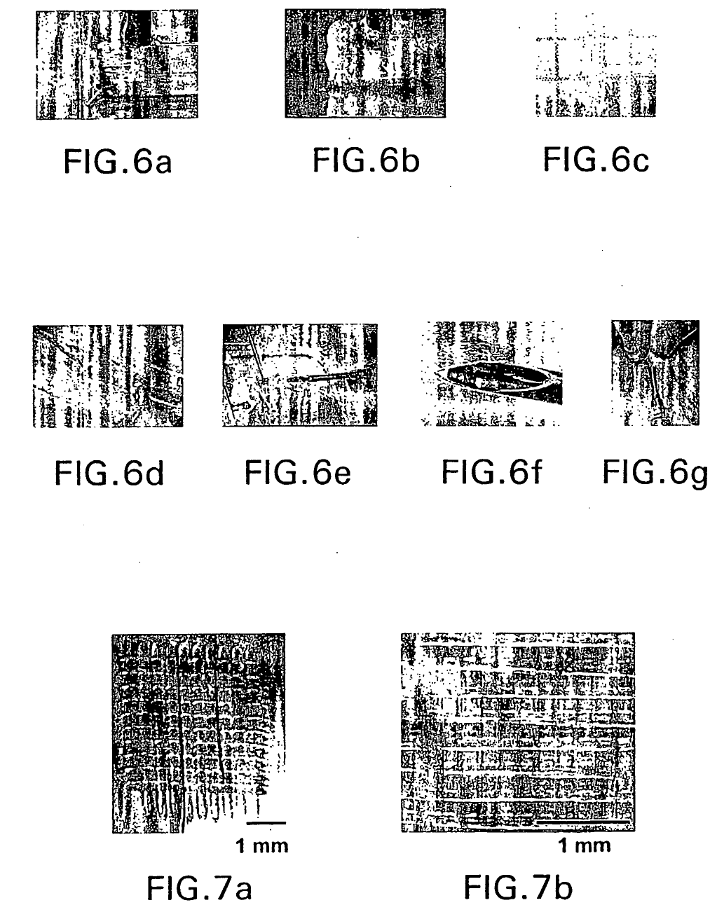

An injectable capsule represents a rectangular box 7×1.4×0.8 mm that can be filled with fibrin glue, urinary bladder mucosa (UBM)/HA mixture, photocurable gelatins, PCL, or another biomaterial of choice “in-line,” utilizing the multiple-head BAT system. In this particular case, the injectable capsule would be filled with the multilayer DCN ETC shown in FIG. 6(b). The capsule deposited on the glass slide can be easily detached and inserted into a special needle for a subcutaneous injection, as shown in FIG. 6(d)-(g).

The injection needle used in these experiments was supplied with a plastic plunger that pushed the capsule out. Injected with due care, the capsule remained undamaged. It is envisaged that subcutaneous injection of the DCN will be required for functionality. One of ordinary skill in the art will understand that such vehicles for enclosing the DCNs of the invention can be made in any convenient shape, e.g., square, rectangular, or other-shaped box, capsular, spherical, ovoid, cylindrical, etc.

Capsules

Capsules such as shown in FIG. 5 and FIG. 6 should keep all elements of the device together for the time necessary for curing or experimental observation. Meanwhile, they should allow cell migration both from outside into the device and vice versa, as necessary. Sensitive and easily soluble materials like collagen-bearing signaling peptides should be protected by the capsule from early erosion. In contrast, structural elements of the capsule can and should work themselves as eroding vehicles for chemoattractants and cytokines to release them in due time. All of these properties can be attained using the “bioconcrete” and “mesh basket” concepts discussed below.

Degradable Mesh

A degradable mesh as shown in FIG. 7 is fabricated by the BAT from such photoreactive materials as PPF, PPF-PEG, or PPTD, or by the solidification of viscous yet volatile solutions of PCL or PLCL. The wire probes show the open channels in FIG. 7(a). These mesh structures will become elements of more-complex devices.

Bioconcrete

Biodegradable mesh structures made from the relatively hard materials named above can become “rebars” in composite blocks wherein the role of “cement” is assigned to soft hydrogels, either natural, such as collagen, HA, ECM, or fibrin glue, or synthetic, such as PEG derivates. Liquid sols deposited on the top of reasonably thick mesh packs will penetrate inside, congealing afterwards. Those composite structures will be able to retain soft gels significantly longer than the exposed gels. Thus, the biodegradability of the natural polymers can be significantly extended in the bioconcrete meshes. Accordingly, these reinforced gels can serve as reliable and long-lasting depots for more-hydrophilic cytokine peptides and other bioactive substances that have a biological or physiological effect on cells or tissue, e.g., chemicals, vitamins, hormones, molecules, proteins, nucleic acid molecules (e.g., plasmid or viral vectors), antigens, and chemokines. In addition to its structural role, the “rebar” materials can be loaded with molecules (e.g., chemoattractants, modulators, or antigens) that require slower release kinetics compared with the molecules encapsulated in the gel (“cement”). For example, hydrophobic chemoattractants and other bioactive substances, such as the chemoattractant fMLP and its derivatives, can be loaded into the rebars. “Bioconcrete” structures can readily incorporate cells provided that the hydrogel “cement” is soft enough to allow cellular motility. Multivehicular systems of nano- and microspheres loaded with cytokines can be comfortably adopted by “bioconcrete” structures to produce an even more developed delivery system.

Mesh Basket

The mesh basket is a combination of the concept of the injectable capsule with that of the multilayered mesh (FIG. 7(a)). Indeed, a rectangular- or honeycomb-grid mesh can become the bottom of the encapsulating box, for which walls will be built in regular LBL fashion.

Platforms for Viability Tests

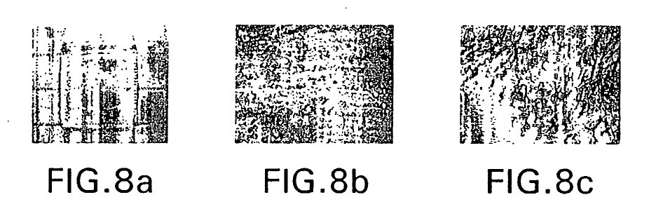

These tests were designed for assessing the viability of cells deposited into various environments, placed onto materials chosen for encapsulation in the DCN, or performing another structural role. The test platforms (also referred as “test-wells”) were built in the 30-mm Petri dishes LBL as square boxes, about 4×4×0.3 mm, with the expanded foundation, as shown in FIG. 8. Cell carriers, such as fibrin glue/HA or ECM/HA composites, were placed in the box with cells either deposited simultaneously or on the top of the whole construct. The medium was carefully poured into the Petri dish to cover the construct.

PPTD and PF-127/PPF-PEG were both used to build the test-wells. Gamma-irradiated nondividing fibroblasts were used as a test culture. The construct has demonstrated viability within 48 hours at 37° C.

Microparticle Controlled Release Strategies

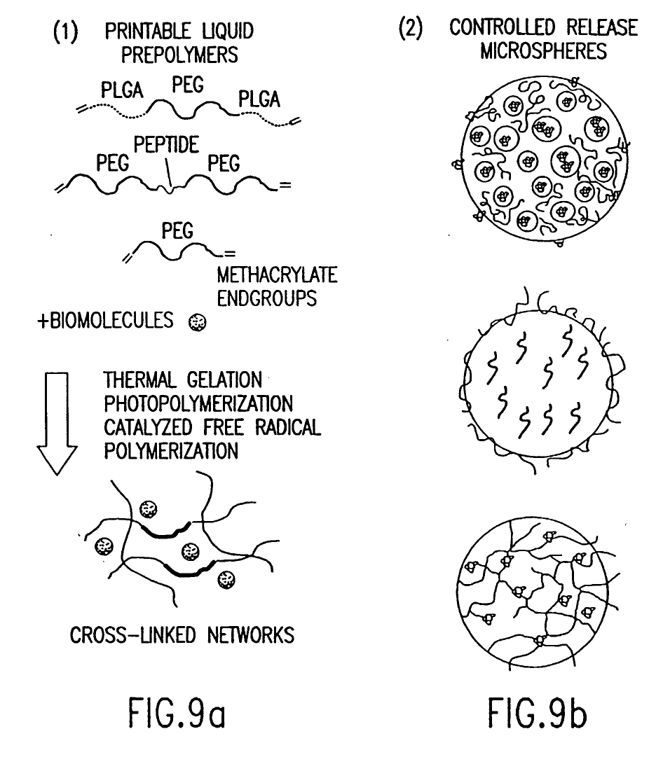

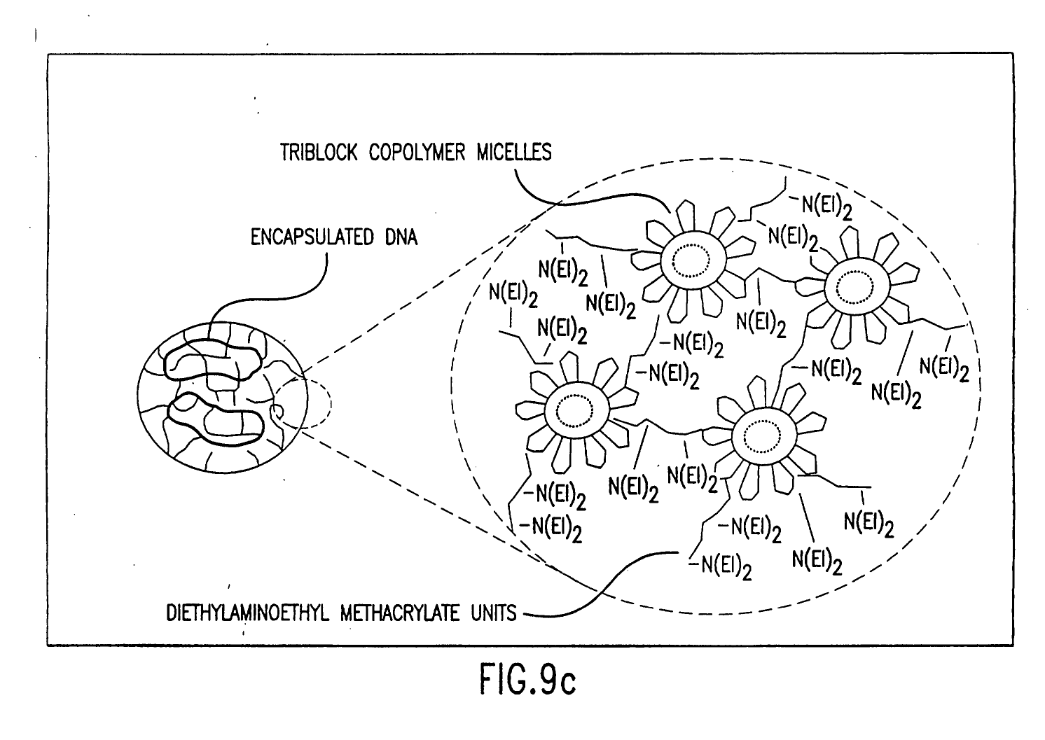

Having presented the base construction scaffold materials, controlled biomolecule release strategies for the DCN are now addressed. Typical synthetic or natural scaffolds capable of multiple molecular-factor delivery can be fabricated from the DCN construction materials shown in FIG. 9. The resulting construct allows sustained biomolecule delivery and maintenance of the biological activity of incorporated and released cytokines, chemokines, antigens, DNA plasmids, peptides, etc. These biomolecules can be incorporated into scaffolds by several approaches as schematically illustrated in FIG. 9. There are generally three distinct types of release matrices: (1) printable biomaterials (e.g., triblock copolymer hydrogels) for the tailored release of proteins; (2) gel-immobilized degradable microspheres for the tailored release of peptides and small-molecule factors; and (3) gel-immobilized hydrogel nanoparticles for the tailored delivery of such biomolecules as plasmid DNA.

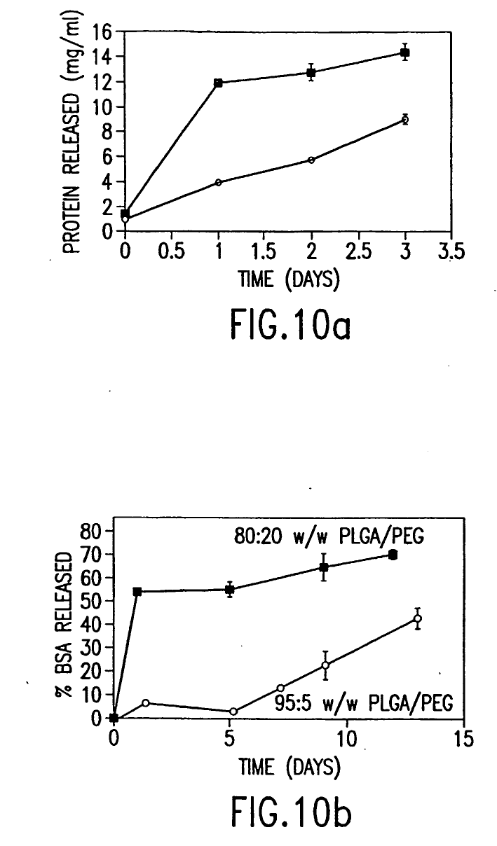

The first methodology involves simply mixing the biomolecules with the base scaffold material and results in a more rapid release, e.g., hours to weeks, as shown in FIG. 10. The base scaffold materials (biomaterials) also provide a matrix for immobilization of microspheres (e.g., PLGA/PEG) and hydrogel nanoparticles within layers of the DCN. As one example, printable aqueous solutions have been developed of the methacrylated PLGA-PEG-PLGA triblock copolymer. These are solidified in situ during printing for either immobilization of microspheres and nanoparticles in desired locations within a specific DCN layer or for direct encapsulation of biomolecular factors within the DCN layer. The triblock copolymer can be printed as a viscous aqueous solution and cured by ultraviolet photopolymerization during printing. Factors may be added to the triblock solution and encapsulated in the hydrogel for controlled release (FIG. 10(a)), or the hydrogel can be used to immobilize PLGA/PEG microspheres or triblock copolymer nanoparticles in a desired location in printed devices. For example, by blending different amounts of the hydrophilic polymer PEG with the more hydrophobic PLGA, release profiles for proteins and peptides from these microspheres can be tailored, as shown in FIG. 10(b). Even though a specific example is provided above on how to tailor the release of proteins from PLGA/PEG nano/microspheres, the general methodology is similar in concept for other biomaterial systems as well.

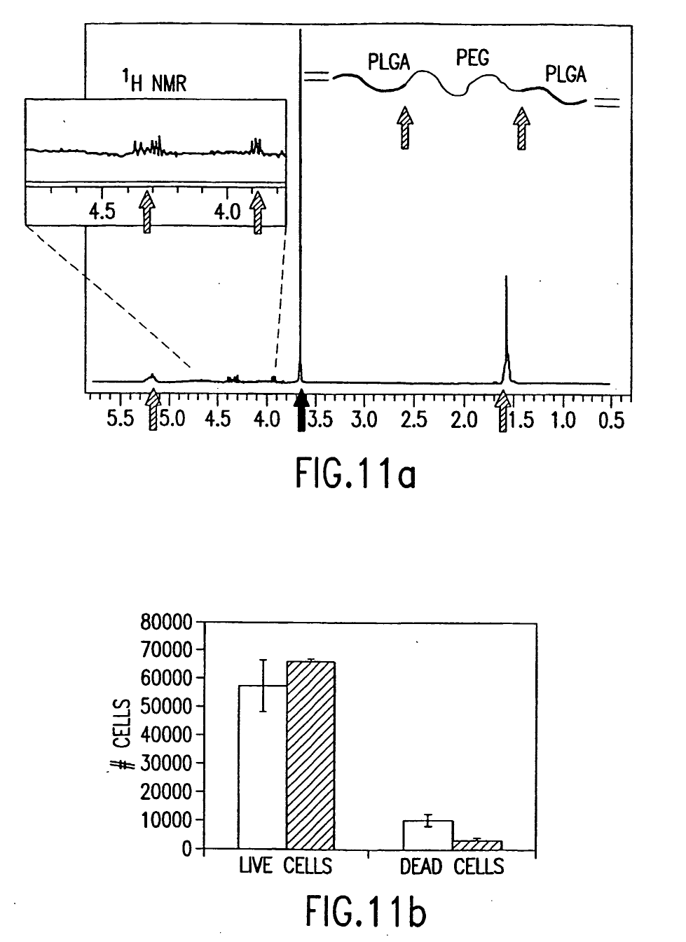

To boost the mechanical strength of natural- or biopolymer-based scaffolds, as well as to provide materials for building biodegradable controlled-release components of the drug delivery devices described herein, triblock copolymers composed of a central PEG block with short terminal PLGA blocks were developed. As shown in the nuclear magnetic resonance (NMR) data in FIG. 11(a), these are end-capped with methacrylate or acrylate double bonds, allowing polymerization of these materials into a network hydrogel. Variation of the relative lengths of the PLGA and PEG blocks allows the degradation rate of the hydrogel to be tuned over a broad range and release of encapsulated factors to occur over a few days or up to a month.

Hydrogels of the triblock copolymer are ideal for controlled release of the chemotactic proteins, since these matrices can be formed under mild aqueous conditions (room-temperature photo-polymerization) and encapsulate high concentrations of the protein in a local site in the scaffold. Degradation of the gel will control release of the protein over time. Printing of the triblock copolymer has been tested using the BAT and it was found that it could be readily printed into 3D constructs. Toxicity of these materials towards dendritic cells was tested in vitro, as shown in FIG. 11(b). No significant difference in viability was observed between DC's exposed to 100 mg of hydrogel or controls with no exposure for 24 hours.

Another approach involves pre-encapsulating the biomolecules in microspheres, and then embedding these microspheres into the host scaffold (see FIG. 9(a)). Another approach involves attaching the biomolecule to the surface of the microsphere. The last approach involves gel immobilized hydrogel nanoparticles. These “particle” based technologies are discussed next. The microspheres and nanoparticles are complementary technologies (summarized in FIG. 12), both of which are “printable” formulations.

The following discussion provides exemplary methods in which to fabricate the “particles” and how they are incorporated for temporal control of various biomolecules. The first of these controlled-release components are PLGA/PEG blend microspheres like those shown in the optical micrograph (OM) of FIG. 12(a). These are prepared by a double-emulsion technique similar to that reported previously (Ref. 17), and can be used to encapsulate drugs in microspheres having sizes tunable from <1 μm to ˜100 μm. PLGA has been used for many years as a controlled-release material due to its relative biocompatibility and hydrolysis rate. As shown in FIG. 10(b), addition of different amounts of water-soluble PEG in the microspheres allows the release profile of encapsulated factors to be varied dramatically, due to the formation of microscopic channels in microspheres as PEG dissolves.

The second exemplary component developed for delivery of factors from the DCN are biodegradable hydrogel nanoparticles, prepared using a crosslinkable triblock copolymer and a cationic pH-sensitive co-monomer, as illustrated in FIG. 12(b). The nanogel colloid proved miscible with many of the scaffold materials listed in Table IV. In mixing the nanogel with collagen, thrombin, and fibrinogen, no significant denaturation of the proteins was observed; the fibrinogen/thrombin system completely retained activity.

These nanogel particles are designed in particular for the delivery of DNA to cells effectively: (1) encapsulation in the nanoparticles should protect DNA from rapid degradation by extracellular DNAses; (2) the particles are designed to be readily endocytosed by cells; and (3) the particles have been engineered to aid the release of DNA into the cytosol by providing a “proton-sponge” effect that can disrupt endosomes, triggered by the reduced pH in these intracellular compartments. The A-B-A triblock is composed of a central PEG B block (4,600 Da) with A blocks composed of PLGA (50:50 w/w lactide:glycolide, each 1,150 Da), and each end of the triblock is capped with a methacrylate group after the approach of Sawhney et al. (Ref. 18). Nanoparticles were synthesized by photopolymerization of a water/oil/water double emulsion. In model DNA delivery experiments, an aqueous solution of pVRC gp120 HIV DNA—250 μL of 0.05 g/mL poly(vinyl alcohol) containing 1.6 mg/mL DNA—was added to 1 mL of dichloromethane (Aldrich) containing 200 mg methacrylated PLGA-PEG-PLGA, 350 μL 2-diethylaminoethyl methacrylate, and 4 mg phenylbis(2,4,6-trimethylbenzoyl)phosphine oxide photoinitiator. The mixture was sonicated to form an emulsion. This primary emulsion was then added to 20 mL of aqueous 0.05 g/mL poly(vinyl alcohol) and sonicated for 30 s to form the second emulsion. The emulsion was subsequently polymerized by exposing the rapidly stirring solution to ultraviolet (365 nm, ˜10 mW/cm²) for 3 minutes. The solution was stirred continuously for 2 h to evaporate dichloromethane from the particles. Particles thus obtained were purified by passing through a 0.2-μm filter followed by concentration in a 50 kDa centriprep concentrator (Amicon) and separation from free monomer using a PD10 desalting column (Amersham Pharmacia). The particles can be fluorescently labeled using rhodamine methacrylate or fluorescein isothiocyanate methacrylate. The pendant amine groups within the gel particle provide pH sensitivity; these groups become charged at reduced pH, causing an electrostatically driven swelling of gel particles. A scanning electron micrograph (SEM) of nanoparticles obtained by this process is shown in FIG. 12(c). Plasmid DNA can be encapsulated in these particles, as illustrated by the gel electrophoresis of DNA recovered from lysed particles (FIG. 12(d)), and the particles are readily internalized by DC's (FIG. 12(e)). This is an important finding for the DCN layers, which can be used to deliver DNA plasmids.

As discussed in the previous paragraph, an alternative to using traditional molecular factors has been recently introduced. The approach combines the concepts of gene therapy and bioengineering. Instead of administering cytokines or chemokines directly, which leads to major dosing and side-effect issues, it is possible to deliver genes that encode those molecules to target cells in vivo. The genes are part of a plasmid, a circular piece of DNA constructed for this purpose. The surrounding cells (phagocytotic cells such as DC's) take up the DNA and treat it as their own. They turn into tiny factories, churning out the cytokines (factors) coded for by the plasmid. Because the inserted DNA is “free-floating,” rather than incorporated into the cells' own DNA, it eventually degrades and the factors cease to be synthesized. It has been demonstrated in animals that 3D biodegradable polymers spiked with plasmids will release that DNA over extended periods and simultaneously serve as a scaffold for new tissue formation. The DNA finds its way into adjacent cells as they migrate into the polymer scaffold, an idea that will be tried for the cytokine depot proposed herein. The cells then express the desired proteins/cytokines. This technique makes it possible to control cytokine release more precisely and over a much longer period to avoid any possible systemic effects.

These biomolecular delivery approaches may be combined by mixing one factor with microspheres containing a pre-encapsulated second factor to provide multiple protein delivery with a distinct release rate for each. The mixed natural or synthetic scaffold and PLGA microspheres will easily fuse to form a continuous, homogeneous matrix.

Examples of Antigens for Use in DCNs

The DCNs of the invention can be used to treat or prevent infectious diseases. One of ordinary skill in the art will understand that the DCNs of the invention can be used to vaccinate subjects against any known infectious agent. Examples of infectious agents that cause disease, along with examples of antigens that can be used in the DCN to vaccinate against these pathogens, include, but are not limited to: human immunodeficiency virus (gp120 protein); malaria (MSP1, AMA1, PfEMP1); tuberculosis (antigen 85 A/B, ESAT-6 and heat shock protein 60); influenza (HA, NA); hepatitis B virus (HBeAg); see, e.g., Letvin N L, Barouch D H, Montefiori D C. Prospects for vaccine protection against HIV-1 infection and AIDS. Annu Rev Immunol. 2002; 20:73-99; Richie T L, Saul A. Progress and challenges for malaria vaccines; Nature. 2002 Feb. 7; 415(6872):694-701; Andersen P. TB vaccines: progress and problems. Trends Immunol. 2001 March; 22(3):160-8.

The DCNs of the invention can also be used to treat or prevent various cancers, by vaccinating the subject with one or more antigens that will stimulate an immune response against the tumor. Many tumor antigens are known, and one of ordinary skill in the art will know how to select the appropriate antigen for treating or preventing a specific tumor. Examples of types of cancer and examples of antigens that can be used in the DCN to vaccinate against these cancers, include, but are not limited to: melanoma (MART-1, MAGE-1, tyrosinase, gp100, GAGE family); cervical cancer (human papilloma virus antigens E6 and E7); Burkitt's lymphoma (EBV antigens); CML (bcr-abl fusion product); colorectal, lung, bladder, head and neck (mutant form of p53); B cell non-Hodgkin's lymphoma and multiple myeloma (Ig idiotype); prostate cancer (PAA, PSA, PSMA); thyroid cancer (thyroglobulin); liver cancer (alpha-fetoprotein); breast and lung (her-2/neu); colorectal, lung, breast (CEA); colorectal, pancreatic, ovarian, lung (muc-1); many cancers (telomerase, oncogenic mutations in RAS, cdk4, p53 or other oncogenes tumor suppressors); see, e.g., Fong L, Engleman E G. Dendritic cells in cancer immunotherapy. Annu Rev Immunol. 2000; 18:245-73.

In addition, one of skill in the art will appreciate that there is a large number of adjuvants that are known to modulate dendritic cell activity (e.g. Tlr ligands and cytokines such as IL-2, IL-7, IL-15, IL-13, TNF-alpha, CD40 activators; see Table III). The skilled artisan will understand that one or more of these modulators can be used in the DCN to stimulate DC maturation for effective anti-pathogen or anti-tumor immunity. See, e.g., Pardoll D M. Spinning molecular immunology into successful immunotherapy. Nat Rev Immunol. 2002 April; 2(4):227-38.

The Various Layers of an Exemplary DCN Described in Detail

Having discussed the base scaffold biomaterials to construct the DCN layers, the list of candidate materials used to construct the “capsule” housing the DCN construct for subcutaneous injection, methods to improve the construction properties of natural polymers, schemes to reduce the degradation rate of natural polymers, and micro- and nanoparticle strategies for controlled release of the biomolecules, a detailed examination is now provided of the individual layers of the heterogeneous DCN ETC and the biomolecules that are embedded in each layer to induce a specific response and/or functionality. The digital printing BAT can fabricate all the layers of the DCN by depositing them in LBL mode to form a 3D heterogeneous ETC.

First Layer: Monocyte Chemoattractant Layer 0110

The first layer is a monocyte chemoattractant layer 0110 as shown in FIG. 1. This layer attracts monocytes from the blood to the DCN. The reason for attracting monocytes is that they are a more plentiful cell source in the blood as opposed to DC's—monocytes comprise approximately 30% of the white blood cells, whereas DC's are only about 0.5% of the total. The more abundant monocytes make statistical interaction with the DCN more likely.

The monocytes are attracted by a number of chemokines such as fMLP, MIP3-α, and MCP-1, MCP-2, MCP, MIP1α, MIP1β, RANTES, HCC-1, HCC-2, HCC-4, MPIF-1, C5a, b-defensin to name a few. The concentration ranges for these chemokines are from 1 picomolar tol millimolar (e.g., in the picomolar and/or micromolar range, e.g., 1-10 pM; 10-100 pM; 100 pM-1 μM; 1-10 μM; 10-100 μM, etc.).

Samples have been fabricated to test chemotactic behavior. The Sample below is intended to be exemplary only, as one of ordinary skill in the ail will understand that many other combinations can be used for the biomaterial scaffold as well as for the types and combinations of chemokines. These chemokines can be built into the scaffold matrix during fabrication, or they are surface-immobilized on nano/microparticles that are added to the scaffold material during fabrication, or they can be embedded in the nano/microparticles that are added to the scaffold material during fabrication. For the specific example provided herein, the samples contain fMLP-O-Me (the methyl ester of fMLP) as the chemoattractant that is embedded in the scaffold matrix, and has been built in LBL mode from fibrin glue components. Generally, the scheme is as such:

The first layer containing fibrinogen or thrombin component (the names for the solutions are “Fibro” and “Thrombo,” respectively), is deposited to make a square patch 8×8 mm, ≈200 μm thick.

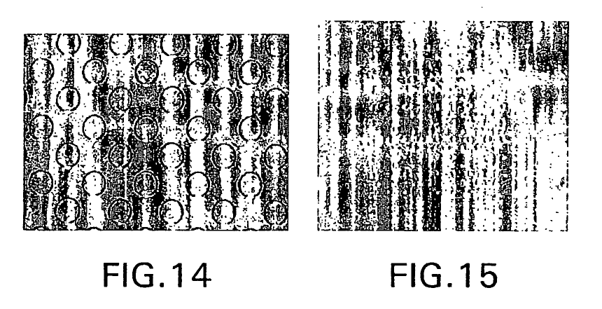

A solution of fMLP-O-Me is injected deep into the patch in a checkerboard mode (FIG. 14). Multiple short-time injections are made that cover homogeneously the central 5.1×5.1 mm part of the patch, leaving the margins free (FIG. 15). (These solutions could also be encapsulated in biocompatible microspheres.)

A second 8×8 mm layer of the counter-component, i.e., “Thrombo” if the first solution was “Fibro” and vice versa, is deposited to cover the chemokine. Multiple layers can be constructed to make the layers thicker if needed. The samples are left in covered Petri dishes in the refrigerator overnight or over a weekend to dry them out.

The solutions used were:
(1) “Fibro”: 80 mg/mL human fibrinogen in distilled water+0.3% HA.
(2) “Thrombo”: 22 mg/mL human thrombin in distilled water+0.5% HA; no Ca²⁺ has been added.
(3) fMLP-O-Me: 5 mM in (33% glycerol+67% dimethylsulfoxide, v/v).

The injection pattern used was:
162 dots in a shifted checkerboard mode;
linear dot-to-dot distance 600 μm;
total weight of solution deposited ≈1.2 mg (FIG. 15).
   Second Layer: Monocyte Differentiation Layer 0120

The second layer is one that differentiates the more abundant monocytes into iDC's in the DCN. DC's are the “professional” APC's and hence the most important cell type to the DCN. The biomolecular factors that induce differentiation are well known and established in the literature. Several candidates include interferon-α, flt3L, or GM-CSF, IL-4, IL-3, TGFb, IL-15, IL-7, IL-2 proteins as the differentiation factors directly embedded in the scaffold matrix or surface-immobilized on biocompatible microspheres such as PLGA.

Third Layer: Antigen Presentation Layer 0130

Having differentiated the monocytes to iDCs, the next stage is to load the desired antigens into these iDCs. Antigens embedded into the scaffold matrix or surface-immobilized on micro or nano-particles are methods in which antigen presentation to the iDCs occurs. Such antigens could be libraries of expressed peptides (1 nanogram-1 milligram; e.g., 10-100 ng; 100 ng-1 μg; 10-100 μg; 100 μg-1 mg, etc.), recombinant peptides or proteins, DNA plasmids to express antigens, etc. Solid polymer microspheres for antigen delivery can be composed from such biodegradable polymers as PLGA, polyanhydrides, polyphosphazenes, PCL, and their copolymers by single- or double-emulsion fabrication methods. Gel particles can be prepared from biodegradable networks, e.g., cross-linkable PLGA-PEG-PLGA or PCL-PEG-PCL block copolymers or PEG-peptide-PEG copolymers with an enzymatically degraded peptide sequence (Ref. 19); or nondegradable networks, e.g., ionically crosslinked alginate or chitosan, polymethacrylates, or crosslinked dextrans. Antigens can be encapsulated in gel/solid polymer particles, immobilized to the surface, or both. Antigens engulfed by DC's are readily loaded onto class II MHC's for presentation to CD4⁺ helper T cells, but do not load class I MHC's for presentation to CD8⁺ killer T cells. Because CD8⁺ T cells are likely critical for immune responses to persistent infections and for fighting cancer, the DCN must provide a mechanism for loading class I MHC's with chosen antigens. To achieve this, incorporation of micro- and nanogel particles formed using the degradable triblock copolymers to deliver antigens intracellularly to DC's are employed. These particles, when engulfed by DC's, are designed to disrupt endosomes by swelling at the reduced endosomal pH within DC's and/or through a “proton sponge” effect (Ref. 20), causing release of antigen into the cytosol, where it can be loaded onto class I MHC's.

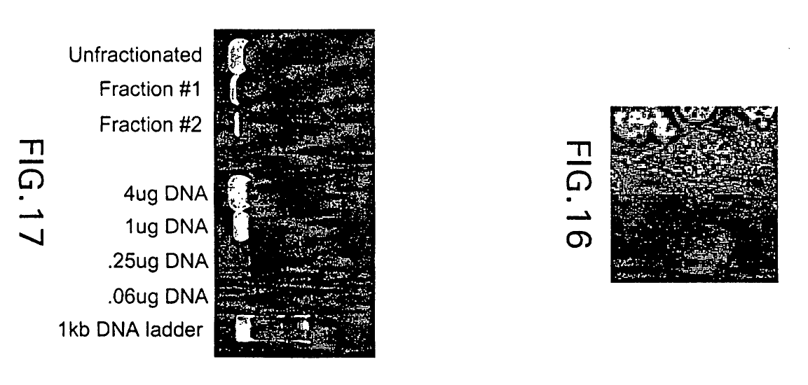

Gel particles encapsulating the model protein antigen ovalbumin have been prepared by photopolymerizing an emulsified solution of the triblock copolymer, protein, and a cationic amino monomer, as illustrated in FIG. 12(b). Initial experiments confirm that protein-loaded gel particles are readily taken up by DC's. Shown in FIG. 16 are fluorescence/brightfield micrographs from an example DC after 1 hour exposure to a nanoparticle suspension. Particles are distributed throughout the cell body. Fluorescence was stable in cells for several days in culture, supporting the hypothesis that these may serve a dual rule as tracers for antigen-exposed cells in vivo. Particle uptake at the densities shown did not have any acute toxicity for DC's (viability equivalent to controls that were not exposed to particles). The maximal protein antigen loads that can be incorporated in the particles, what sizes can be prepared, and how degradation rates of the particles can be tuned by composition variation are currently being assessed.

The literature provides ample precedent for particle-based class I antigen loading in DC's. It has been demonstrated (Ref. 21) that antigen adsorbed to the surface of latex beads (and many other types of particles) leads to cross-priming and class I antigen loading on DC's. This method of antigen delivery is 100-1000 times more potent than simply exposing DC's to free protein antigen. However, the fact that protein is only adsorbed to particle surfaces is a serious limitation, because only a tiny amount of protein can be delivered. Using the nanoparticles described herein, protein, peptides, or nucleic acid is distributed throughout the particle volume, allowing potentially 1000-fold more Ag to be delivered.

Finally, successful digital printing of nanogels with hyaluronic acid has been demonstrated, i.e., no agglomeration of the nanogels was observed. This shows the demonstration that the antigen presentation layer of the DCN construct can be easily built.

The ideal vaccine would deliver a simple, low-cost antigen constitutively to DC's. One way to increase the potency of antigen presentation would be to use the DCN to transfect in situ DC's with DNA causing DC's to produce antigen for themselves. This general concept was discussed earlier. To consider this option in our device design, we tested DNA encapsulation with triblock gel particles and found that DNA can be incorporated similar to proteins. Shown in FIG. 17 (and in FIG. 12) is an ethidium-bromide-stained gel electrophoresis result on DNA extracted from nanoparticles, along with DNA standards for comparison. The “unfractionated” lane shows DNA both inside and outside particles prior to purification, and “fraction 2” shows DNA that was entrapped in particles (≈40 μg).

In some cases, DNA plasmids may express intracellular antigens for presentation on MHC class I; in other examples, they may express secreted proteins that DCs will carry to and produce in the draining lymph nodes. Secreted proteins may be fusions of DC-binding ligands. For example, fusion of Ig or complement C3 with an antigen allows antigens to enter the MHC class I pathway, even when delivered outside of the cell (Regnault A, Lankar D, Lacabanne V, Rodriguez A, Thery C, Rescigno M, Saito T, Verbeek S, Bonnerot C, Ricciardi-Castagnoli P, Amigorena S. Fcgamma receptor-mediated induction of dendritic cell maturation and major histocompatibility complex class I-restricted antigen presentation after immune complex internalization. J Exp Med. 1999 Jan. 18; 189(2):371-80). In addition, DNA plasmids may express any protein ligands that may modulate dendritic cell maturation for use in particular disease states (see section below describing layer 4).

Fourth Layer: Maturation and DC Chemoattractant Layer 0140

The fourth layer of the DCN ETC is comprised of a chemoattractant layer to attract IDC's further into the scaffold and of a signal to further mature the DC's. The DCs are attracted by potentially a number of chemokines such as fMLP, MIP3-α, and MCP-1, MCP-2, MCP, MIP1α, MIP1β, RANTES, HCC-1, HCC-2, HCC-4, MPIF-1, C5a, b-defensin to name a few (Zlotnik A, Yoshie O. Chemokines: a new classification system and their role in immunity. Immunity. 2000 February; 12(2): 121-7; Yang D, Chertov O, Bykovskaia S N, Chen Q, Buffo M J, Shogan J, Anderson M, Schroder J M, Wang J M, Howard O M, Oppenheim J J. Beta-defensins: linking innate and adaptive immunity through dendritic and T cell CCR6. Science. 1999 Oct. 15; 286(5439):525-8; Sozzani S, Sallusto F, Luini W, Zhou D, Piemonti L, Allavena P, Van Damme J, Valitutti S, Lanzavecchia A, Mantovani A. Migration of dendritic cells in response to formyl peptides, C5a, and a distinct set of chemokines. J. Immunol. 1995 Oct. 1; 155(7):3292-5). The concentration ranges for these chemokines are from 1 picomolar to 1 millimolar (e.g., in the picomolar and/or micromolar range, e.g., 1-10 pM; 10-100 pM; 100 pM-1 μM; 1-10 μM; 10-100 μM, etc.). The concentration of the chemokines in this layer will need to be less than that of the monocyte attractant layer (e.g., at least 2-fold less; at least 5-10-fold less; at least 10-25-fold less; at least 25-50-fold less; at least 50-100-fold less). The lower concentration creates an attractive gradient within the DCN to move the DC's through the various layers.

Chemokines

Peptide or protein-entrapping microspheres composed of PLGA either alone or blended with PEG have been tested for controlled release of chemoattractants in the DCN. These microspheres are formed by a simple double- or single-emulsion process (for proteins and peptide encapsulation, respectively) and can be prepared with sizes ranging from <1 μM to >100 μm diameters. By blending different amounts of the hydrophilic polymer PEG with the more hydrophobic PLGA, release profiles for proteins and peptides from these microspheres can be tailored, as shown in FIG. 10(b).

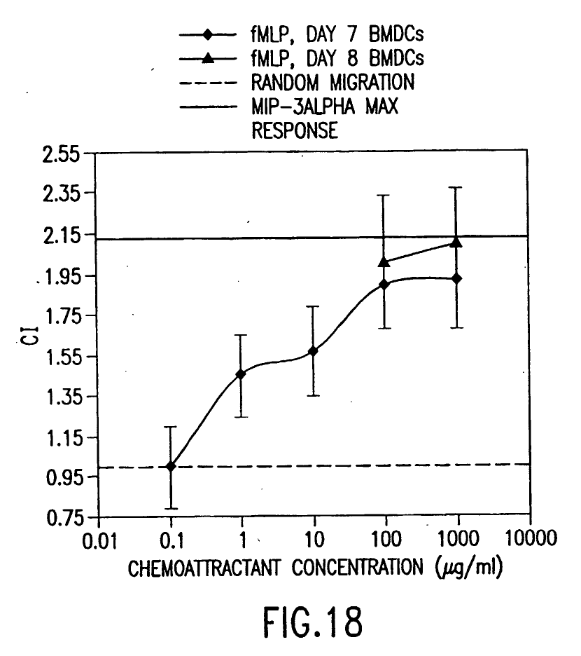

The formyl peptide fMLP (formyl-Met-Leu-Phe) chemoattractant has been studied in addition to the protein chemokine MIP-3α. The formyl peptide is a bacterial byproduct that attracts DC's to sites of infection. It has been reported in the literature to be attractive for iDC's in mice and humans. Tests were carried out with this material and found that the peptide attracted bone marrow-derived dendritic cells with a maximal potency comparable to MIP-3α (FIG. 18). For these experiments, DC's were placed on the top of a migration filter containing 5-μm pores with a reservoir of fMLP (or MIP-3α) at the indicated concentration on the other side. After 90 minutes, the number of cells migrating in response to the chemoattractants was counted and compared to controls. In FIG. 18, CI is the chemotaxis index, defined as (number of migrated cells in chemokine)/(number of migrated cells in control without chemokine).

The literature reports CI up to ≈5 max for bone-marrow-derived dendritic cells (BMDC's), but this experiment was carried out on late-stage DC cultures (DC's are starting to mature on Day 7) and the culture was not purified, thus a significant contamination with neutrophils is likely present; thus the real CI is possibly higher. Of importance is that fact that high concentrations of fMLP appear to give comparable results to MIP-3α (which in previous experiments are found gave maximal migration at 1 μg/mL, in line with literature reports). Having found that fMLP does chemoattract DC's, controlled-release PLGA microspheres to deliver this agent for chemoattraction in the DCN device is the preferred embodiment.

Use of fMLP has numerous advantages over MIP-3α: (1) It is a 3-mer peptide, inexpensive and commercially available in large quantities, hence much more economical both for experiments and from the standpoint of viable commercial vaccines; (2) since it is only a peptide, there are no concerns with stability within microspheres/gels or shelf life; and (3) as it is very hydrophobic, it is readily encapsulated in PLGA microspheres. (A hydrophobic, low-molecular-weight cargo is the “ideal” case for microsphere encapsulation and release.) PLGA microspheres are used to deliver this agent as its low molecular weight makes it unfeasible to slow its release in hydrogels (it will diffuse out essentially unimpeded).

Maturation Signal

The DC state is an important parameter in determining the nature of the immune response (Ref. 21). The most basic DC states described in the literature are the immature and mature states: immature DC's are poised to capture antigens but lack the requisite accessory signals for T-cell activation, while mature DC's have a reduced capacity for antigen uptake but an exceptional capacity for T-cell stimulation. Immature DC's, contrary to previous assumptions, are not ignored by the immune system and can lead to tolerance by inducing IL-10-producing, antigen-specific regulatory T cells. Maturing DC's redistribute MHC class II molecules to the plasma membrane and upregulate surface co-stimulatory molecules, MHC class I, and T cell adhesion molecules. Mature DC's also modify their profile of chemokine receptors, which enable homing to lymphoid organs (Ref. 22).

Differences in the expression of MHC, adhesion, costimulatory, and other molecules as well as differences in cytokine secretion further subdivide mature DC states and can influence the nature of the immune response. In a recent study the different adaptive immune responses produced by lipopolysaccharide (LPS) from different bacteria (Escherichia coli and Porphyromonas gingivalis) were linked to the different cytokine expression profiles in mature DC's (Ref. 22) (Ref. 22). E. coli LPS induced a T-helper cell (T_{H}1)-like response, while P. gingivalis LPS induced a T_{H}2-like response. The DC expression of three cytokines, IL-12, IL-6, and tumor necrosis factor (TNF)-α, was measured. IL-12 was induced only in the DC's of E. coli LPS-treated mice; expression of IL-6 and TNF-α was similar in DC's from both treatment groups. This finding is consistent with other reports showing that mature, IL-12-producing DC's transform CD4-expressing T-helper cells into IFN-γ-producing T_{H}1 cells and lead to cell-mediated immunity, while DC's in the presence of IL-4 induce T cells to differentiate into T_{H}2 cells and lead to humoral immunity. Most importantly, understanding the effects of different DC states allows rational intervention; it is this understanding that is exploited in the DCN. The DCN puts the DC in the right state to activate a desired immune response.

Modulating the Dendritic Cell State

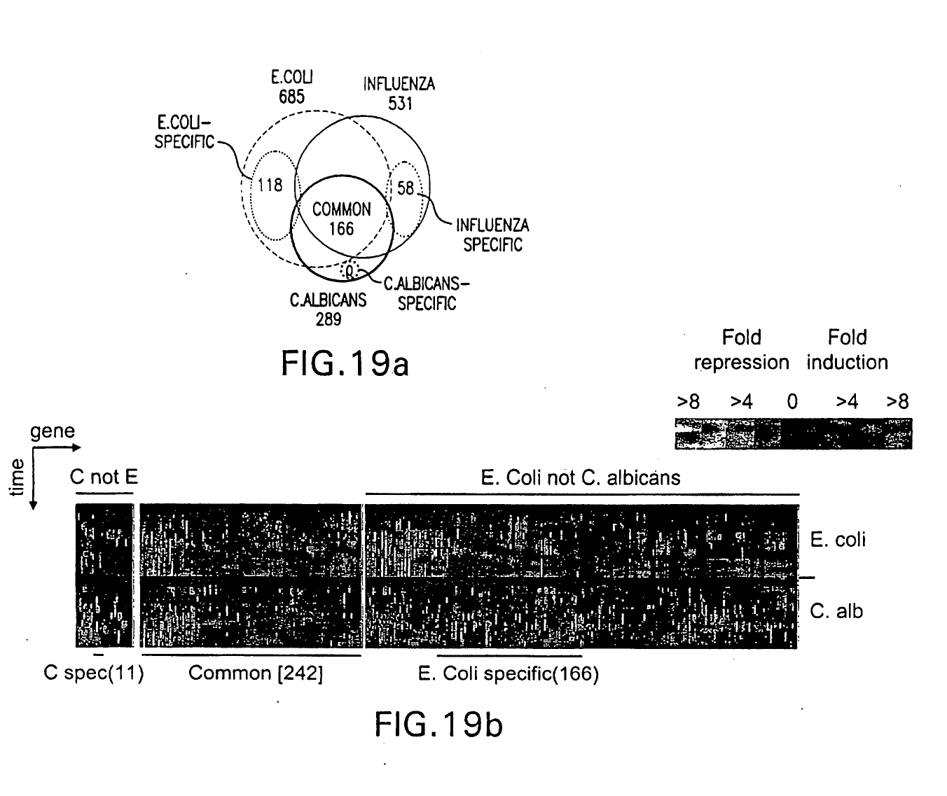

Prior to recent work conducted at the Whitehead Institute (WI), the downstream target genes induced in DC's by different pathogens had not been fully determined. To systemically explore the gene expression profile of DC's, WI exposed human-monocyte-derived DC's to a diverse set of organisms and compounds: (1) the Gram-negative bacterium E. coli, and its cell-wall component LPS; (2) the fungus Candida albicans, and its cell-wall-derived mannan; and (3) the RNA virus influenza A, and its double-stranded RNA. DC's were cultured with pathogens or their components and RNA expression was measured using oligonucleotide microarrays. FIG. 19 shows an analysis of pathogen-regulated genes as well as a comparison of mRNA expression levels in response to two pathogens. Image A shows overlapping sets of E. coli, C. albicans, and influenza-regulated genes; Image B shows a representation of mRNA expression levels at 0, 1, 2, 4, 8, 12, and 24 hours in response to E. coli and C. albicans. The colored bars represent the ratio of hybridization measurements between corresponding time points in the pathogen and control medium profiles.

Of the ≈6,800 genes studied, a total of 1,330 genes changed their expression significantly upon encounter with one of the pathogens or components. Such a large-scale change in gene expression demonstrates that DC's can undergo dramatic transformations in their cellular phenotype. DC maturation, therefore, should not be simply defined by the modulation of a standard set of markers. Table V illustrates the wide functional variety in genes regulated.

The WI genome-wide analysis of DC gene expression reveals many genes with potential immunostimulatory roles. For example, anti-apoptotic genes may extend the lifetime of infected DC's, and matrix metalloproteases may allow cytokine processing and DC migration to lymph nodes. In addition, many genes with undefined roles in DC function were also identified, including signaling molecules, transcription factors, and adhesion molecules. Since E. coli differentially up-regulated most innate immune response genes on the array, including neutrophil-attracting chemokines (see Table V), WI tested the in vitro migration of neutrophils toward conditioned cell-cultured medium collected from DC's exposed to E. coli, influenza, or control medium. WI found significant migration with E. coli treatment versus little to no migration in the influenza or control treatments. Thus, DC state modulation has consequences for the type of elicited immune response. It is this DC state modulation that is controlled by the DCN and in part makes this TE vaccine unique.

The DC states, based on DC gene expression profiles, allow the rational optimization of the modulation of DC's for the DCN. Using this knowledge of DC states and gene expression increases the specificity and potency of immune responses against pathogens.

Table III displays examples of ligands for use in modulation of DC's in the biomaterial scaffold for the maturation signals. These signals are embedded in the scaffold matrix, or are surface immobilized on microspheres embedded in the scaffold, or are embedded in the micro/nanoparticles that are added to the scaffold. The antigen-loaded DC's encounter the layer that contains these candidate biomolecular state modulators. In addition, these maturation ligands may also be coupled to antigens covalently or non-covalently. Or, in the case of protein ligands, may be fused genetically and expressed as a fusion protein.

In addition, for pathogens that evade immunity, it may be possible to reverse this evasion with appropriate inhibitors. And finally, in the case of autoimmune diseases, ligands that are inhibitors of dendritic cell activation will be essential to turn responses toward tolerance; or inhibitors of stimulatory ligands may reduce autoimmunity (such as Tlr9 inhibitors: Reference: Leadbetter E A, Rifkin I R, Hohlbaum A M, Beaudette B C, Shlomchik M J, Marshak-Rothstein A. Chromatin-IgG complexes activate B cells by dual engagement of IgM and Toll-like receptors. Nature. 2002 Apr. 11; 416(6881):603-7).

Fifth Layer: Optional Symmetry Layer 0150

The fifth layer is an optional layer largely based on symmetry of the DCN ETC. The fifth layer is not necessary for DCN functionality. However, it may be a comprised of a number of a various material and constituent formulations, and serve the following optional functions such as: (a) a thin scaffold material, with no specific biomolecules, to control the release of the DC's; or (b) an additional antigen-presenting layer. Thus, if iDC's statistically encounter the DCN, they will phagocytosize the antigens and then encounter the chemoattractant and maturation signal layers to form fully mature DC's. In this case the motility of the DC's is upward in FIG. 1.

Sixth Layer: Optional Encapsulation Layer

The sixth layer is also optional depending on the release characteristics or the fragility of the DCN ETC. The sixth layer, or really encapsulating layer, is a biocompatible “capsule” such as that shown in FIG. 5 and FIG. 6. The encapsulating layer can optionally be loaded with signal molecules (e.g., chemoattractants, antigens, monocyte or DC modulators, etc.)

Variations in Layer Construction of the DCN

The above discussion of the various layers is illustrative only, as several of the functionalities of the various layers can be combined together. For instance, layers 0110 and 0120 (monocyte attractant layer and monocyte differentiation layer) are illustrated as distinct layers, but could alternatively be constructed as one layer. The important aspects of the DCN are what the construct does; it does not necessarily have to use distinct layers to accomplish its functionality.

Also, only certain layers of the DCN construct are necessary to induce an enhanced immune response. For example, instead of providing separate layers for monocyte attraction and differentiation, as described above, one can simply attract iDC's to the construct and load them with chosen antigens and appropriate state modulators. Similarly, it may only be necessary to have an iDC depot to illicit an enhanced immune response. In this case, the only layer required would be the DC chemoattractant layer 0110. Thus, one of ordinary skill in the art will understand that variations, permutations, and combinations of the layers are included in the present invention.

Constructing the Dendritic Cell Node by Other Means

As described previously, the DCN can be constructed by a LBL deposition process using such digital printing processes as that afforded by the BAT. In LBL construction, each layer is subsequently built on top of the previous layer. However, due to the nature and relative lack of restrictions on shape or size of the DCN, it could also be constructed, even in layered fashion, through other methods. Two examples are illustrated below.

Folded Constructs

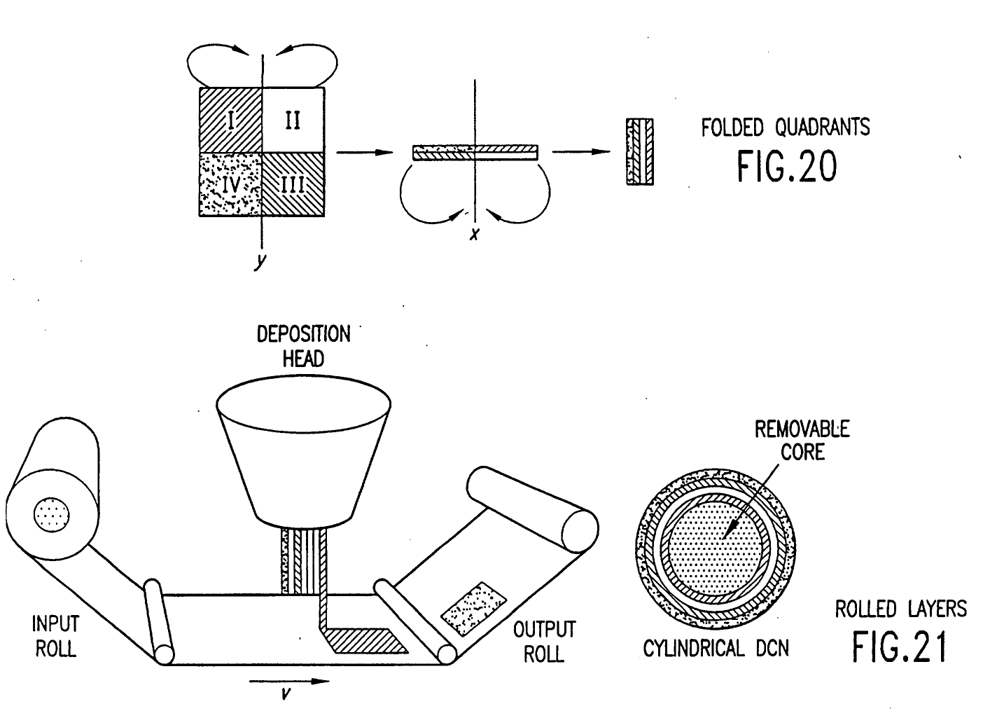

One alternative method by which to construct a layered DCN is to “sandwich” the membranes. Specifically, a TE membrane biomaterial could be designed to include various individually engineered borders or sections, e.g., quadrants. In each border or section, the appropriate various biomolecular factors are added, then the whole structure is folded so as to create a 3D structure, as shown in FIG. 20. For example, in a four-quadrant folded structure, biocompatible chemokine microspheres could be placed in the upper-right quadrant II, nanogels containing DNA plasmids could be placed in the lower-right quadrant III, and structural materials could be placed in quadrants I and IV, which become the outermost layers.

These engineered quadrants could be constructed in a number of ways by using the BAT, such other digital printing tools as electrosprays and inkjets, or such manual printing tools as micropipets. After the quadrants are constructed, the membrane is then folded in such a way that the various layers are still distinct and in the proper order from the topmost to the bottommost layers. In FIG. 20, this is accomplished by folding the originally flat xy-plane structure around the y axis, then by folding the resultant yz-plane structure around the x axis. (In the figure, thickness is exaggerated to show the layered structure.) For this to be possible, the membrane must be thin, pliable, and flexible, besides biocompatible. Candidates for such membranes could include ECM sheets, fibrin sheets, or collagen sponge scaffolds.

Roll-to-Roll Constructs

Another method by which to fabricate a DCN in layered fashion is to use a roll-to-roll process in essence comparable to the web-handling techniques widely used in printing and other industries. The basic scaffold or substrate material should be thin, pliable, and flexible yet biocompatible; suitable materials include ECM, fibrin, or collagen.

The advantage of modern computer-controlled web-handling techniques is that the substrate sheet moves from the feed or input roll to the uptake or output roll at a known rate. Such parameters as the angular velocities of the two rolls and the resultant thickness of the layers deposited onto the output roll can be calculated and controlled. Meanwhile, the motion of the sheet past the writing heads and table determines the rate at which the active components of the DCN must be deposited.

As the substrate moves past, various dispensing units, such as electrosprays, inkjets, BAT printing elements, micropipets, or other tools can be used to “print” the various biomolecular components onto the substrate in conveyor-belt fashion. Once these printed regions reach the output roll, the individual printed layers can be compiled to make the overall 3D structure with the separate layers still resolved, which in this case will have cylindrical symmetry, as is illustrated in FIG. 21.

Immune Modulation by the DCN

In one specific example, the present invention provides a method by which DCN-hosted DC's offer a solution to the previous problem of developing a malaria vaccine that can initiate T-cell responses at one stage and B-cell responses at others. It is now apparent that the key to an effective malaria vaccine is that it must initiate both T_{H}1 and T_{H}2 responses, leading to the stimulation of cytotoxic T lymphocytes (CTL's) and antibody-producing B lymphocytes. Previous vaccine research has focused upon only one of these pathways, T_{H}1 producing CTL's or T_{H}2 producing antibodies. Existing vaccines do not work well because of this limitation of focus and temporal control. The DCN is the only present technology that allows the initiation of T_{H}1 responses at certain stages and T_{H}2 at others. Ordinarily, the T_{H}1 and T_{H}2 pathways cannot be induced simultaneously by a single conventional vaccine because the T_{H}1 cytokines block the T_{H}2 pathway and vice versa. However, the novel aspect of the DCN operates by making it possible to induce these different immune responses at different times, on demand. The DCN can also be used to modulate DCs to block the T_{H}2 pathway, thereby blocking allergic responses.

The way the type of immune response can be controlled via the DCN ETC is by controlling the degradation rates of the scaffold material and the means of its construction via a layer-by-layer growth mechanism. For example, some of the ETC layers could be built to have largely a T-cell response (e.g., by incorporating IL-12, IL-2, or IFN-γ in the scaffold matrix during fabrication) followed by layers that would induce a B-cell response (e.g., by incorporating IL-4 and IL-10 in the TE scaffold during fabrication), etc.

For autoimmune diseases, it is possible to construct an DCN with antigens that are found as targets of autoimmune responses (e.g. insulin or GAD for diabetes, myelin basic protein for multiple sclerosis, acetylcholine receptor for myasthenia gravis, etc.) and state modulators that would turn dendritic cells into tolerizing cells (e.g. vitamin D, IL-10 or other tolerizing agents), thus leading to the reduction of the autoimmune response due to T and B cells. (Yoon J W, Jun H S. Cellular and molecular pathogenic mechanisms of insulin-dependent diabetes mellitus. Ann N Y Acad. Sci. 2001 April; 928:200-11; MS, Stinissen P, Medaer R, Raus J. Myelin reactive T cells in the autoimmune pathogenesis of multiple sclerosis. Mult Scler. 1998 June; 4(3):203-11; De Baets M, Stassen M H. The role of antibodies in myasthenia gravis. J Neurol Sci. 2002 Oct. 15; 202(1-2):5-11; S. Gregori, N. Giarratana, S. Smiroldo, M. Uskokovic, and L. Adorini A 1 {alpha}, 25-Dihydroxyvitamin D3 Analog Enhances Regulatory T-Cells and Arrests Autoimmune Diabetes in NOD Mice Diabetes, May 1, 2002; 51(5): 1367-1374; M. D. Griffin, W. Lutz, V. A. Phan, L. A. Bachman, D. J. McKean, and R. Kumar Dendritic cell modulation by 1alpha, 25 dihydroxyvitamin D3 and its analogs: A vitamin D receptor-dependent pathway that promotes a persistent state of immaturity in vitro and in vivo. PNAS, Jun. 5, 2001; 98(12): 6800-6805).

Other examples of tolerizing agents that can be used in the DCN include aspirin, steroidal or non-steroidal anti-inflammatories, ATP, TGF-β, ligands or activators of the following receptors: SIR-P, CD36, mer or DC-SIGN; as well as several other ligands shown in Table III (troglitazone, bradykinin, etc). Alternatively, by ensuring that DCs attracted to the DCN are immature (i.e. by not providing any activators in the DCN construct), tolerance will ensue. Finally, by attracting plasmacytoid DCs specifically, it should be possible to induce tolerance with or without a maturation-inducing stimulus in the DCN. In summary, there are many ways to block dendritic cell maturation and ensure that T and B cells are not optimally activated and undergo tolerance (anergy, deletion or differentiation into regulatory T cells) instead of activation. (See, e.g., Yin et al. The anti-inflammatory agents aspirin and salicylate inhibit the activity of IkB kinase-beta. Nature 1998, 396:77; Webster et al. Neuroendocrine Regulation of Immunity. Annu. Rev. Immunol. 2002, 20:125-63; la Sala et al. Extracellular ATP Induces a Distorted Maturation of Dendritic Cells and Inhibits Their Capacity to Initiate Th1 Responses. J. Immunol., 2001, 166: 1611-1617; Latour et al. Bidirectional negative regulation of human T and dendritic cells by CD47 and its cognate receptor signal-regulator protein-alpha: down-regulation of IL-12 responsiveness and inhibition of dendritic cell activation. J Immunol 2001 Sep. 1; 167(5):2547-54; Britta et al. A role for CD36 in the regulation of dendritic cell function. PNAS 2001 vol. 98(15): 8750-8755; Cohen et al. Delayed Apoptotic Cell Clearance and Lupus-like Autoimmunity in Mice Lacking the c-mer Membrane Tyrosine Kinase. J. Exp. Med 2002 Volume 196, Number 1, Jul. 1, 2002 135-140; Teunis et al. Mycobacteria Target DC-SIGN to Suppress Dendritic Cell Function. J. Exp. Med. 2003 Volume 197, Number 1, Jan. 6, 2003

7-17; Dhodapkar and Steinman. Antigen-bearing immature dendritic cells induce peptide-specific CD8(+) regulatory T cells in vivo in humans. Blood 2002 Jul. 1; 100(1):174-7; Gilliet and Liu. Generation of human CD8 T regulatory cells by CD40 ligand-activated plasmacytoid dendritic cells. J Exp Med 2002 Mar. 18; 195(6):695-704).

Exemplary DCN Constructions

The following provides examples of combinations of monocyte chemokines, differentiation proteins, antigens, maturation ligands, and chemoatttrants that can be used to construct the DCNs of the invention. These examples are not intended to be limiting, as it will be clear to one of ordinary skill in the art that any appropriate combination of monocyte chemokines, differentiation proteins, antigens, maturation ligands, and chemoatttrants as described herein or as known in the art or later discovered can be used to construct the DCNs of the invention.

Components of a DCN for Treating or Preventing an HIV Infection

1. Monocyte chemokine layer: fMLP, and/or MIP3α, to attract monocytes from the blood to the DCN.
2. Monocyte differentation protein layer: flt3L, INF-α to differentiate monocytes into dendritic cells.
3. Antigen layer: either recombinant gp120 protein (Genbank NC_{—}001802) or a DNA plasmid version with gp120 fused to the Fc portion of human Ig in order to get efficient B cell responses as well as T cell responses (gp120-Fc fusion will bind to the follicular dendritic cells that present antigens to B cells and stimulate B cells antibody production).
4. Maturation layer ligands and chemoattractant: CpG oligo for the ligand, and fMLP or MIP3α for the chemokine. The chemokine concentration of this layer should be less than that of layer 1 (at least two-fold less).
5. Antigen layer: same as 3.
   Components of a DCN for Treating or Preventing Diabetes
1. Monocyte chemokine layer: MIP3α.
2. Monocyte differentation protein layer: flt3L.
3. Antigen layer: insulin-B (Genbank Accession No. J00265) or GAD (Genbank Accession No. M74826).
4. Maturation layer ligands and chemoattractant: Vitamin D or IL-10 for the ligands, and MIP3α for the chemokine. The chemokine concentration of this layer should be less than that of layer 1 (e.g., at least two-fold less).
5. Antigen layer: same as 3.
   Components of a DCN for Treating or Preventing Multiple Sclerosis
1. Monocyte chemokine layer: MIP3α.
2. Monocyte differentation protein layer: flt3L.
3. Antigen layer: myelin basic protein (Genbank Accession No. X17286).
4. Maturation layer ligands and chemoattractant: Vitamin D or IL-10 for the ligands, and MIP3α for the chemokine. The chemokine concentration of this layer should be less than that of layer 1 (e.g., at least two-fold less).
5. Antigen layer: same as 3.
   Components of a DCN for Treating or Preventing Myasthenia Gravis
1. Monocyte chemokine layer: MIP3α.
2. Monocyte differentation protein layer: flt3L.
3. Antigen layer: acetylcholine receptor alpha subunit (Genbank Accession No. y00762).
4. Maturation layer ligands and chemoattractant: Vitamin D or IL-10 for the ligands, and MIP3α for the chemokine. The chemokine concentration of this layer should be less than that of layer 1 (e.g., at least two-fold less).
5. Antigen layer: same as 3.
   Advantages of the DCN

Use of an ETC to harbor chemokines, cytokines, modulators, and/or antigens for the DCN, with or without exogenously-added DC's, provides a hub to attract and “train” DC's to present a chosen antigen, as well as a biocompatible harboring site designed to keep the DC's alive. The DCN provides the proper microenvironment/spatial control to modulate and program the DC's to induce a specific immune response. Moreover, the biodegradable natures of the scaffold and the embedded biomolecules, microspheres, or nanoparticles containing the biomolecules provide temporal control over any specific arm of the immune system and/or release of specific cytokines or chemoattractants.

Use of the DCN to stimulate or tolerize the immune system has numerous advantages, as has been discussed herein. For example, the DCN concentrates DCs by attracting them to a small volume in the body (e.g. subcutaneously), and enhances antigen delivery to DCs by providing large amounts of antigen where DCs are attracted and concentrated. The DCN also enhances DNA plasmid or viral-based delivery of antigens by concentrating DCs and thus effectively increasing specific delivery of DNA and viral particles to DCs rather than other cell types (e.g. fibroblasts, endothelial cells, muscle cells, keratinocytes). Moreover, use of nanoparticles for antigen presentation greatly enhances the amount of antigen that is presented to the DCs.

In addition, the DCN modulates the state of concentrated dendritic cells uniformly using protein or non-protein ligands (including small molecules) that regulate the activity of specific receptors or proteins expressed in dendritic cells. Moreover, the DCN can employ DNA vaccines or viral vectors to express genes that can modulate the DC state.

DCNs can contain bioconcrete in any or all layers, to reduce the degradation rate of biomaterials within the DCN. The bioconcrete can contain bioactive substances, such as (but not limited to) chemicals, peptides or polypeptides, anti-virals, for controlled drug release. The bioconcrete can also contain microspheres and/or nanoparticles containing such bioactive substances.

Incorporation by Reference

Throughout this application, various publications, patents, and/or patent applications are referenced in order to more fully describe the state of the art to which this invention pertains. The disclosures of these publications, patents, and/or patent applications are herein incorporated by reference in their entireties, and for the subject matter for which they are specifically referenced in the same or a prior sentence, to the same extent as if each independent publication, patent, and/or patent application was specifically and individually indicated to be incorporated by reference.

### OTHER EMBODIMENTS

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### LITERATURE CITED

1. J. Banchereau and R. M. Steinman, “Dendritic Cells and the Control of Immunity,” Nature 1998, 392 (6673), 245.
2. N. C. Fernandez, A. Lozier, C. Flament, P. Ricciardi-Castagnoli, D. Bellet, M. Suter, M. Perricaudet, T. Tursz, E. Maraskovsky, and L. Zitvogel, “Dendritic Cells Directly Trigger NK Cell Functions: Cross-Talk Relevant in Innate Anti-Tumor Immune Responses In Vivo,” Nat. Med. 1999, 5 (4), 405.
3. M. V. Dhodapkar, J. Krasovsky, R. M. Steinman, and N. Bhardwaj, “Mature Dendritic Cells Boost Functionally Superior CD8⁺ T-Cell in Humans Without Foreign Helper Epitopes,” J. Clin. Invest. 2000, 105 (6), R9.
4. T. Kumamoto, E. K. Huang, H. J. Paek, A. Morita, H. Matsue, R. F. Valentini, and A. Takashima, “Induction of Tumor-Specific Protective Immunity by In Situ Langerhans Cell Vaccine,” Nat. Biotechnol. 2002, 20 (1), 64.
5. J. Banchereau and R. M. Steinman, “Dendritic Cells and the Control of Immunity,” Nature 1998, 392 (6673), 245.
6. J. Banchereau, F. Briere, C. Caux, J. Davoust, S. Lebecque, Y.-J. Liu, B. Pulendran, and K. Palucka, Ann. Rev. Immunol. 2000, 18, 767.
7. R. C. Fields, K. Shimizu, and J. J. Mule, “Murine Dendritic Cells Pulsed with Whole Tumor Lysates Mediate Potent Antitumor Immune Responses In Vitro and In Vivo,” Proc. Natl. Acad. Sci. USA 1998, 95 (16), 9482.
8. D. C. Reid, “Dendritic Cells and Immunotherapy for Malignant Disease,” Br. J. Haematol. 2001, 112 (4), 874.
9. J. I. Mayordomo, T. Zorina, W. J. Storkus, L. Zitvogel, C. Celluzzi, L. D. Falo, C. J. Melief, S. T. Ildstad, W. M. Kast, A. B. Deleo, et al., “Bone Marrow-Derived Dendritic Cells Pulsed with Synthetic Tumour Peptides Elicit Protective and Therapeutic Antitumour Immunity,” Nat. Med. 1995, 1 (12), 1297.
10. L. Zitvogel, J. I. Mayordomo, T. Tjandrawan, A. B. DeLeo, M. R. Clarke, M. T. Lotze, and W. J. Storkus, “Therapy of Murine Tumors with Tumor Peptide-Pulsed Dendritic Cells: Dependence on T Cells, B7 Costimulation, and T Helper Cell 1-Associated Cytokines,” J. Exp. Med. 1996, 183 (1), 87.
11. M. Gunzer, S. Janich, G. Varga, and S. Grabbe, “Dendritic Cells and Tumor Immunity,” Semin. Immunol. 2001, 13 (5), 291.
12. J. Banchereau, A. K. Palucka, M. Dhodapkar, S. Burkeholder, N. Taquet, A. Rolland, S. Taquet, S. Coquery, K. M. Wittkowski, N. Bhardwaj, L. Pineiro, R. Steinman, and J. Fay, “Immune and Clinical Responses in Patients with Metastatic Melanoma to CD34⁺ Progenitor-Derived Dendritic Cell Vaccine,” Cancer Res. 2001, 61 (17), 6451.
13. J. Banchereau, B. Schuler-Thurner, A. K. Palucka, and G. Schuler, “Dendritic Cells as Vectors for Therapy,” Cell 2001, 106 (3), 271.
14. C. Klein, H. Bueler, and R. C. Mulligan, “Comparative Analysis of Genetically Modified Dendritic Cells and Tumor Cells as Therapeutic Cancer Vaccines,” J. Exp. Med. 2000, 191(10), 1699.
15. C. Brunner, J. Seiderer, A. Schlamp, M. Bidlingmaier, A. Eigler, W. Haimerl, H. A. Lehr, A. M. Krieg, G. Hartmann, and S. Endres, “Enhanced Dendritic Cell Maturation by TNF-Alpha or Cytidine-Phosphate-Guanosine DNA Drives T Cell Activation In Vitro and Therapeutic Anti-Tumor Immune Responses In Vivo,” J. Immunol. 2000, 165 (11), 6278.
16. M. V. Dhodapkar, R. M. Steinman, J. Krasovsky, C. Munz, and N. Bhardwaj, “Antigen-Specific Inhibition of Effector T Cell Function in Humans After Injection of Immature Dendritic Cells,” J. Exp. Med. 2001, 193 (2), 233.
17. E. C. Lavelle, et al. Vaccine 1999, 17(6), 512.
18. A. S. Sawhney, C. P. Pathak, and J. A. Hubbell, “Interfacial Photopolymerization of Poly(ethylene glycol)-based Hydrogels upon Alginate-poly(L-lysine) Microcapsules for Enhanced Biocompatibility,” Biomaterials 1993, 14 (13), 1008.
19. West and Hubbell. Macromolecules 1999, 32 (1), 241.
20. O. Boussif, et al. Proc. Natl. Acad. Sci. USA 1995, 92, 7297.

21. I. Mellman and R. M. Steinman, “Dendritic Cells: Specialized and Regulated Antigen Processing Machines,” Cell 2001, 106 (3), 255.

TABLE I
Abbreviations and Symbols

| | | | |
|---|---|---|---|
| DCN | Dendritic Cell Node | nD | n-Dimensional (n = 3) |
| APC | Antigen-Presenting Cells | NKC | Natural Killer Cell |
| BAT | Biological Architectural Tool | NMR | Nuclear Magnetic Resonance |
| BMDC | Bone-Marrow-Derived Dendritic Cell | OM | Optical Micrograph/scope |
| BW | Biological Warfare | PBS | Phosphate-Buffered Saline |
| CAD | Computer-Aided Design | PCL | Poly(caprolactone) |
| CAM | Computer-Aided Manufacturing | PEG | Poly(ethylene glycol) |
| CI | Chemotaxis Index | PF-127 | Pluronic F-127 |
| CTL | Cytotoxic T Lymphocyte | PLCL | Poly(lactide-co-caprolactone) |
| DC | Dendritic Cell | PLGA | Poly(lactide-co-glycolide) |
| DNA | Deoxyribonucleic Acid | PPF | Poly(propylene fumarate) |
| ECM | Extracellular Matrix | PPTD | (PLGA-co-PEG)-triblock-dimethacrylate |
| ETC | Engineered Tissue Construct | SEM | Scanning Electron Micrograph/scope |
| HA | Hyaluronic Acid | TE | Tissue-Engineered |
| HIV | Human Immunodeficiency Virus | T_{H}1 | Helper T Cell, Type I |
| iDC | Immature Dendritic Cell | T_{H}2 | Helper T Cell, Type II |
| IFN | Interferon | Tlr | Toll-Like Receptor |
| LBL | Layer-by-Layer | Tₘ | Temperature, Melting Point (° C.) |
| LPS | Lipopolysaccharide | UBM | Urinary Bladder Mucosa |
| MHC | Major Histocompatibility Complex | WI | Whitehead Institute |
| MIT | Massachusetts Institute of Technology | x, y, z | Cartesian Coordinates (m) |

TABLE II
Two Modes of the DCN

| Scaffolds without Cells | Scaffolds with Cells |
|---|---|
| Load scaffold with: | 1) Start from dendritic cells pulsed |
| 1) chemokines/cytokines; | with desired antigens or modulators. |
| 2) target antigens; | 2) Load scaffolds with cells and |
| 3) modulators for desired immune | cytokines. |
| response (immunity or tolerance). | |

TABLE III
Examples of Dendritic Cell Modulators

| Receptor | Modulator | Functional Category |
|---|---|---|
| Serotonin | Serotonin | Neurotransmitter |
| Receptor β1 | | |
| GABA A | GABA | Neurotransmitter |
| Receptor β2 | | |
| Bradykinin Receptor | Bradykinin | Pain/Inflammation |
| Somatostatin | Somatostatin | Neuropeptide |
| Receptor 5 | | |
| Vasopressin | Vasopressin | Vasopressor Peptide |
| Receptor 1B | | |
| PPARγ Receptor | 15dPGJ(2), Troglitazone | Endocrine Regulation |
| FK506 Binding | FK506 | Immunomodulator |
| Protein | | |
| Vitamin D Receptor | Vitamin D | — |
| Purine Receptor | ATP, adenosine | Endogenous ligand |
| P2x4 | | |
| TGFb Receptor II | TGFb | Cytokine |
| IL-2R | IL-2 | Cytokine |
| IL-4R | IL-4 | Cytokine |
| IL-7R | IL-7 | Cytokine |
| IL-13Ra1 | IL-13 | Cytokine |
| IL-15 | IL-15 | Cytokine |
| 4-1BB | 4-1BB Ligand | Immunomodulator |
| CD40 | CD40 ligand | Immunomodulator |
| RANK | RANK ligand | Modulator of DC |
| | | survival |
| Tlr-4 | LPS, hsp6, hsp70, | Pathogen Component |
| | hyaluronic acid fragment, | |
| | saturated fatty acids | |
| Tlr-3 | dsRNA (e.g. polyI:C) | Pathogen Component |
| Tlr-9 | CpG DNA | Pathogen Component |
| Tlr-2 | Bacterial Lipoproteins, | Pathogen Component |
| | hsp60, SP-A, | |
| | peptidoglycan, GPI anchor | |
| | from T. cruzi. | |
| Tlr-5 | Flagellin | Pathogen Component |
| Tlr-1 | Mycobacterial lipoprotein | Pathogen Component |
| | triacylated lipopeptides | |
| Tlr-6 | Mycobacterial | Pathogen Component |
| | lipoproteins, lipoteichoic | |
| | acid, peptidoglycan | |
| Tlr-8 | Resiquikopd, imiquimod | Synthetic compounds |

TABLE IV
Examples of Scaffold Materials

| Material | Summary | Rating |
|---|---|---|
| Poly(propylene fumarate-co- | PPF-PEG still represents the best combination of | *** |
| ethylene glycol) (PPF-PEG) | constructive properties. | |
| PEG-diacrylates and | These produce weak hydrogels that are improper for cell | * |
| -dimethacrylates | attachment. They can be used for cytokine or antigen | |
| | delivery. | |
| Poly(ethylene oxide) | Poly(ethylene oxide) gels are soluble but have minimal | * |
| | constructive properties. | |
| Poly-4-hydroxybutyrate | This strong, natural, biodegradable plastic has a very high | * |
| | melting point, Tₘ > 175° C. | |
| Poly(ethylene-co-vinyl acetate) | This strong, non-biodegradable plastic has Tₘ ≧ 100° C. | * |
| (PLGA-co-PEG)-triblock- | PPTD is “friendly” to cells. Its constructive properties are. | *** |
| dimethacrylate (PPTD) | fair, weaker than PPF-PEG. | |
| Pluronic F-127 (PF-127) | This is an excellent shape-former due to inverse-temperature | ** |
| | gelation, and it is a good drug carrier. Combined with PPF- | |
| | PEG, it allows building real 3D constructs. | |
| Extracellular Matrix (ECM), | These are promising cell-carriers, entirely natural and | *** |
| Small Intestine Submucosa, and | biodegradable. They require additional processing | |
| Urinary Bladder Mucosa (UBM) | (homogenization and combination). | |
| Collagen Type I | Produces weak and partially soluble hydrogels; it is cell- | ** |
| | friendly. | |
| Calfskin Gelatin | This is a good construction material when deposited hot. It is | ** |
| | cell-friendly and soluble. | |
| Fibrinogen, Thrombin | These form insoluble, stable, biodegradable, and cell-friendly | *** |
| | hydrogel clots. | |
| Hyaluronic Acid (HA) | This universal biological thickener is cell-friendly. It can be | *** |
| | safely combined with any other member of this table. | |
| Poly(caprolactone) (PCL), | Common biodegradable polyesters used extensively in tissue | *** |
| Poly(lactide-co-caprolactone) | engineering applications; require printing from organic | |
| (PLCL), poly(D.L-lactide) | solvent but provide improved mechanical strength to the | |
| | construct. | |
| Photocurable Gelatin | A styrene-derivitized gelatin, this was combined with a | ** |
| | water-soluble carboxylated camphorquinone as a | |
| | photoactivator. The material has proven to be a promising | |
| | biodegradable and biosorbable hydrogel, which adhesiveness | |
| | to living tissues is sometimes superior to that of fibrin glue. | |
| Polyphosphazenes | Biodegradable synthetic polymers that degrade to neutral | |
| | byproducts. | |
| Trimethylene Carbonate | Tough, slowly degrading polymers with good structural | |
| Copolymers | properties. | |
| Scaffold Additives | These are not scaffolds per se, but are components of | *** |
| (Laminin and Fibronectin) | extracellular matrix materials that may be necessary for cell | |
| | proliferation and viability. | |

TABLE V
Functional Categories of Genes Differentially Regulated

| | | | | |
|---|---|---|---|---|
| Neutrophil | GAN | EC | CA | IA |

INNATE

| | | | | |
|---|---|---|---|---|
| i18 | Y00787 | +++ | + | + |
| gro1 | X54489 | +++ | + | ± |
| gro2 | M57731 | +++ | + | ± |
| gro3 | X53800 | ++ | ± | ± |

Inflammation

| | | | | |
|---|---|---|---|---|
| infa | X02910 | ++ | + | + |
| il1b | X04500 | +++ | + | + |
| i16 | X04602 | ++ | + | + |
| il1a | M28983 | + | − | − |
| gcsf | X03656 | ++ | − | − |
| mip1b | M69203 | ++ | + | ++ |
| mip3a/larc | U64197 | ++ | ± | ± |
| mip3b/elc | U77180 | + | ± | ± |
| bf | L15702 | ++ | + | ± |

Prostaglandin/Leukotrienc

| | | | | |
|---|---|---|---|---|
| ptgir | D38128 | ++ | + | + |
| ptger4 | L28175 | − | − | + |
| cox2 | U04636 | ++ | + | + |

ADAPTIVE
T Cell-Th1

| | | | | |
|---|---|---|---|---|
| il12b/p40 | M65290 | ++ | + | − |
| itac | U59286 | + | + | ++ |
| mig | X72755 | + | + | ++ |
| inp10 | X02530 | + | + | ++ |
| ifnb1 | V00535 | + | − | ++ |
| ifna2 | J00207 | − | − | + |
| ifna13 | J00210 | − | − | +++ |
| ifna14 | V00533 | − | − | + |
| ifna16 | M28585 | − | − | + |

T Cell-Th2

| | | | | |
|---|---|---|---|---|
| tarc | D43767 | ++ | + | ± |
| mdc | U83171 | + | + | ± |

T Cell Stimulation

| | | | | |
|---|---|---|---|---|
| 41bbL | U03398 | ++ | − | ± |
| slam | U33017 | +++ | + | + |
| cd86 | U04343 | + | + | + |
| icam1 | M24283 | ++ | + | ++ |
| ebi3 | L08187 | ++ | + | − |

Antigen Presentation

| | | | | |
|---|---|---|---|---|
| b2m | J00105 | ++ | − | ++ |
| Lmp10 | X71874 | + | + | ± |

B cell

| | | | | |
|---|---|---|---|---|
| pbef | U02020 | +++ | + | + |

IMMUNE RECEPTOR

| | | | | |
|---|---|---|---|---|
| il15ra | U316284 | ++ | ± | + |
| il7r | M29696 | ++ | + | + |
| il2r | X01057 | + | ± | − |
| il4r | X52425 | + | + | ± |
| gmcsfr | X17648 | + | ± | − |
| il3r | D49410 | + | + | − |
| 41bb | U03397 | +++ | ± | ++ |
| infr2 | M32315 | ++ | − | − |
| il13ra1 | Y10659 | ++ | ++ | − |
| Cd155 | M24406 | +++ | − | − |
| Cd83 | Z11697 | ++ | ++ | ++ |

IMMUNE TRANSCRIPTION

| | | | | |
|---|---|---|---|---|
| nfkb p52 | S76638 | ++ | ± | + |
| nfkbp50 | M58603 | ++ | ± | ++ |
| nfkb p65 | L19067 | + | + | + |
| nfkb re1B | M83221 | + | ± | − |
| stat5a | U43185 | ++ | + | − |
| stat4 | L78440 | ++ | + | − |
| stat3 | L29277 | + | − | ± |
| irf2 | X15949 | + | − | + |
| irf4 | U52682 | + | ± | ± |
| isgf3 | M87503 | + | ± | + |
| csda | M24069 | ++ | − | − |

GLYCOLYSIS AND ENERGY

| | | | | |
|---|---|---|---|---|
| enol | M14328 | + | − | d |
| Pk3 | X56494 | ++ | − | ± |
| Tpi | J04603 | + | − | − |
| gys | J04501 | + | − | d |
| pgm1 | M83088 | + | ± | d |
| Gk | X69886 | + | ± | − |
| pfkp | D25328 | + | − | − |
| pgk1 | V00572 | + | − | ± |
| g3pdh | X01677 | + | − | ± |
| Ldh1 | X02152 | + | − | ± |
| pgd | U30255 | + | − | ± |
| pgam1 | J04173 | + | + | + |
| Hifla | U22431 | + | − | ± |

APOPTOSIS
Inhibitor

| | | | | |
|---|---|---|---|---|
| Pai2 | M31551 | ++ | − | − |
| Iex-1 | S81914 | ++ | + | − |
| Tax1bp1 | U33821 | + | − | ± |
| Flip | AF005775 | ++ | + | ± |
| bag1 | Z35491 | + | + | ± |
| ciap2 | U37546 | ++ | ++ | + |
| Bc12-a1 | U29680 | ++ | + | + |
| mc11 | L08246 | + | − | + |
| Tau | X56468 | ± | ± | ++ |

Activator

| | | | | |
|---|---|---|---|---|
| casp4 | U28014 | ++ | + | ± |
| Nip3 | U15174 | ++ | ± | − |
| trail | U37518 | + | + | ++ |
| Fas | X63717 | + | + | + |
| casp5 | U28015 | + | − | + |
| bak1 | U16811 | ± | ± | + |
| pmaip1 | D90070 | + | − | ++ |
| casp10 | U60519 | ± | + | + |

GROWTH FACTORS AND RECEPTORS

| | | | | |
|---|---|---|---|---|
| tgfa | X70340 | + | − | − |
| ndp | X65724 | +++ | − | − |
| wnt5a | L20861 | +++ | ± | − |
| aclivinba | X57579 | +++ | ++ | + |
| p2x4 | AF000234 | + | + | − |
| vdr | J03258 | + | ± | − |

TISSUE REMODELING

| | | | | |
|---|---|---|---|---|
| mmp9 | J05070 | ± | + | − |
| mmp7 | L22524 | ++ | − | − |
| mmp3 | X05232 | + | − | − |
| mmp19 | X92521 | + | ± | ± |
| mmp14 | Z48481 | ++ | − | ± |
| mmp12 | L23808 | ++ | + | − |
| mmp10 | X07820 | + | − | − |
| mmp1 | X54925 | + | − | − |
| lad1 | U42408 | + | − | ± |
| cxt12 | U76189 | ± | + | ± |
| collagen- | M55998 | + | − | − |
| a1 | | | | |
| tnr | X98085 | + | − | ± |

CELL STRESS

| | | | | |
|---|---|---|---|---|
| mt1g | J03910 | +++ | ± | ± |
| mt1c | M10942 | +++ | ± | ± |
| btg2 | U72649 | ++ | ± | − |
| fth1 | L20941 | ++ | + | − |
| quiescin | L42379 | ++ | ± | − |
| cagb | M26311 | ++ | − | ± |
| ddit1 | M60974 | ++ | − | ± |
| map3k4 | D86968 | ++ | ± | ± |
| mt1l | X76717 | ++ | − | ± |
| mt1h | X64177 | ++ | + | + |
| mt2a | V00594 | ++ | + | + |
| hspa1a | M11717 | ++ | ± | ++ |
| ninj1 | U72661 | ++ | + | ++ |
| sod2 | X07834 | ++ | + | ++ |
| atox1 | U70660 | + | + | − |
| hspa6 | X51757 | + | ± | − |
| krs1 | U26424 | + | ± | − |
| mt1a | K01383 | ++ | − | − |
| mt1f | M10943 | + | − | − |
| rtp | D87953 | ++ | ± | − |
| cyp450db1 | X07619 | + | ± | ± |
| gst12 | U46499 | + | − | ± |
| hsf4 | D87673 | + | − | ± |
| hspa4 | L12723 | + | − | ± |
| dusp1 | X68277 | + | − | + |
| mtf | X78710 | + | − | + |
| hsp70 | U10284 | ± | − | + |
| hsp27 | Z23090 | − | ± | +++ |
| cbr1 | J04056 | ± | + | + |

IMMUNE INHIBITORS

| | | | | |
|---|---|---|---|---|
| mcp1 | S69738 | + | ++ | ++ |
| il10 | U16720 | ++ | − | − |
| hla-e | X56841 | − | − | + |
| gfrp | U78190 | d | − | + |
| ido | M34455 | ++ | ± | ++ |

Table Abbreviations and codes

| | |
|---|---|
| + | Gene expression is up-regulated in response to pathogen. |
| − | Gene expression is not changed. |
| ++, | Gene expression is changed at a higher level relative to other |
| +++ | pathogens that regulate the same gene (each + denotes |
| | increased expression by a factor of ˜2.5). |
| ± | Gene expression is regulated in a subset of donors. |
| d | Gene expression is down-regulated. |
| GAN | Genbank Account Number |
| EC | Escherichia coli |
| CA | Candida albicans |
| IA | Influenza A virus |

1. A dendritic cell node comprising:
a) a biocompatible scaffold material;
b) a chemokine for attracting immature dendritic cells;
c) a chosen antigen; and
d) a maturation signal for dendritic cells.

2-25. (canceled)

26. A method of constructing a dendritic cell node comprising:
a) depositing, onto a substrate, a layer for attracting monocytes into the dendritic cell node;
b) depositing, onto layer (a), a layer for inducing differentiation of the monocytes into immature dendritic cells;
c) depositing, onto layer (b), a layer for presenting a chosen antigen to immature dendritic cells;
d) depositing, onto layer (c), a layer for attracting dendritic cells and inducing maturation of dendritic cells, thereby constructing a dendritic cell node.

27. (canceled)

28. A method of constructing a dendritic cell node comprising:
a) depositing, onto a substrate, a layer for attracting immature dendritic cells into the dendritic cell node;
b) depositing, onto layer (a), a layer for presenting a chosen antigen to the immature dendritic cells; and
c) depositing, onto layer (b), a layer for attracting immature dendritic cells and inducing maturation of the immature dendritic cells; or
d) depositing, onto a substrate, a layer for attracting immature dendritic cells and inducing maturation of the immature dendritic cells;
e) depositing, onto layer (d), a layer for presenting a chosen antigen to the immature dendritic cells; and
f) depositing, onto layer (e), a layer for attracting immature dendritic cells into the dendritic cell node, thereby constructing an dendritic cell node.

29-47. (canceled)
